(19) United States
(12) Patent Application Publication
(10) Pub. No.: US 2025/0275880 A1
(43) Pub. Date: Sep. 4, 2025

(54) A GARMENT ASSEMBLY FOR TRIGGERING, FACILITATING OR RESISTING MOVEMENT

(71) Applicant: DENNISSON TECHNOLOGIES LIMITED, Auckland (NZ)

(72) Inventors: Anvil Banez, Auckland (NZ); Kimberly Banez, Auckland (NZ); Scott Wilson, Auckland (NZ); Thomas Kerr-Phillips, Auckland (NZ); Jamin Martin, Auckland (NZ)

(21) Appl. No.: 18/860,530

(22) PCT Filed: Apr. 29, 2023
(86) PCT No.: PCT/IB2023/054487

§ 371 Date: Oct. 25, 2024

(30) Foreign Application Priority Data
Apr. 29, 2022 (AU) 2022901131

(51) Int. Cl. A61H 1/02; A41D 19/00; G06F 3/01

(52) U.S. Cl. CPC A61H 1/0288; A41D 19/0024; A61H 2201/12; G06F 3/014; G06F 3/016

(57) ABSTRACT

The present invention relates in general to a garment assembly for facilitating or resisting movement of a subject, such as a person, animal, or inanimate object, wearing a garment of the garment assembly. More particularly, the garment assembly comprises one or more smart material actuators that when stimulated mechanically contract and/or expand, thereby creating a force acting on the garment to trigger, facilitate or resist movement of the subject.

### TECHNICAL FIELD

The present invention relates in general to a garment assembly for facilitating or resisting movement of a subject, such as a person, animal, or inanimate object, wearing a garment of the garment assembly. More particularly, the garment assembly comprises one or more smart material actuators that when stimulated mechanically contract and/or expand, thereby creating a force acting on the garment to trigger, facilitate or resist movement of the subject.

### BACKGROUND

Powered exoskeleton or exosuit devices relate to wearable mobile devices that allow for limb movement with increased strength and endurance. Depending on the type of application the exoskeleton or exosuit may be used to facilitate the user's own movements, such as to assist users with impaired mobility, or to act against the user's movements to be used as a rehabilitation or workout tool. While various types of exoskeletons are commercially available there is an ongoing need for further developments, e.g. in terms of minimising the overall system weight, improving the response rate of the system, and/or adding new system functionality.

Hence, an improved garment assembly for triggering, facilitating and/or resisting movement would be advantageous.

### SUMMARY

According to a first aspect a garment assembly for facilitating or resisting movement of a subject, such as a person, animal, or inanimate object, wearing the garment assembly is provided. The garment assembly comprises a garment. Further the garment assembly comprises a first anchor zone, a second anchor zone, each anchor zone acting to secure the garment to a respective body part of the subject, in use. Further, the garment assembly comprises at least one smart material actuator (SMAs) connected between the first anchor zone and second anchor zone, and forming a first group of SMAs. Moreover, the garment assembly comprises a force translation mechanism connecting the first anchor zone to the second anchor zone and comprising the first group of SMAs. Each SMA is arranged to operate in an idle or deactivated state, and an activated state triggered by a non-mechanical stimulus that causes a physical material property change in the associated SMA. Further, the garment assembly comprises an activation unit arranged to transmit said non-mechanical stimulus to each SMA in response to a defined activation sequence.

According to second aspect a garment assembly for facilitating or resisting movement of a subject, such as a person, animal, or inanimate object, wearing the garment assembly is provided. The garment assembly comprises a garment. The garment assembly further comprises a first anchor zone, a second anchor zone, each anchor zone acting to secure the garment to a respective body part of the subject, in use. Further, the garment assembly comprises at least one smart material actuator (SMAs) connected between the first anchor zone and second anchor zone, and forming a first group of SMAs. Moreover, the garment assembly comprises a force translation mechanism connecting the first anchor zone to the second anchor zone and comprising the first group of SMAs. Each SMA is arranged to operate in an idle or deactivated state, and an activated state triggered by a non-mechanical stimulus that causes a geometrical change in the associated SMA. Further, the garment assembly comprises an activation unit arranged to transmit said non-mechanical stimulus to each SMA in response to a defined activation sequence.

According to a third aspect a garment assembly for facilitating or resisting movement of a subject, such as a person, animal, or inanimate object, wearing the garment assembly is provided. The garment assembly comprises a garment having a first anchor zone, a second anchor zone. Each anchor zone acts to secure the garment to a respective body part of the subject, in use. Further, the garment assembly comprises at least two first smart material actuators (SMAs) connected in sequence between the first anchor zone and second anchor zone, and forming a first group of SMAs. A force translation mechanism is provided to connect the first anchor zone to the second anchor zone and comprises the first group of SMAs. Each SMA is arranged to operate in an idle state, and an activated state triggered by a non-mechanical stimulus that causes the associated SMA to mechanically contract. The garment assembly further comprises an activation unit arranged to transmit said non-mechanical stimulus to each SMA individually in response to a defined activation sequence.

According to a fourth aspect a garment assembly for facilitating or resisting movement of a subject, such as a person, animal, or inanimate object, wearing the garment assembly is provided. The garment assembly comprises a garment having a first anchor zone, a second anchor zone, each anchor zone acting to secure the garment to a respective body part of the subject, in use. Further, the garment assembly comprises at least two first smart material actuators (SMAs) connected in sequence between the first anchor zone and second anchor zone, and forming a first group of SMAs. Moreover, the garment assembly comprises a force translation mechanism connecting the first anchor zone to the second anchor zone and comprising the first group of SMAs. Each SMA is arranged to operate in an idle state, and an activated state triggered by a non- mechanical stimulus that causes the associated SMA to mechanically expand. Further, the garment assembly comprises an activation unit arranged to transmit said non-mechanical stimulus to each SMA individually in response to a defined activation sequence.

According to a fifth aspect, a garment assembly for facilitating or resisting movement of a subject, such as a person, animal, or inanimate object, wearing the garment assembly is provided. The garment assembly comprises a garment having a first anchor zone, a second anchor zone, each anchor zone acting to secure the garment to a respective body part of the subject, in use, at least one first smart material actuator (SMAs) connected between the first anchor zone and second anchor zone, and forming a first group of SMAs, a force translation mechanism connecting the first anchor zone to the second anchor zone and comprising the first group of SMAs, wherein the first anchor zone and/or the second anchor zone comprises at least one second SMA, wherein each SMA is arranged to operate in an idle state, and an activated state triggered in response to a non-mechanical stimulus that causes the associated SMA to mechanically contract, wherein said at least second SMA is arranged to mechanically contract in its activated state to secure the garment to the respective body part of the subject or to provide force feedback, and an activation unit arranged to transmit said non-mechanical stimulus to each SMA individually in response to a defined activation sequence.

According to a sixth aspect, a garment assembly for facilitating or resisting movement of a subject, such as a person, animal, or inanimate object, wearing the garment assembly is provided. The garment assembly comprises a garment having a first anchor zone, a second anchor zone, each anchor zone acting to secure the garment to a respective body part of the subject, in use, at least one first smart material actuator (SMAs) connected between the first anchor zone and second anchor zone, and forming a first group of SMAs, a force translation mechanism connecting the first anchor zone to the second anchor zone and comprising the first group of SMAs, wherein the first anchor zone and/or the second anchor zone comprises at least one second SMA, wherein each SMA is arranged to operate in an idle state, and an activated state triggered in response to a non-mechanical stimulus that causes the associated SMA to mechanically expand, wherein said at least second SMA is arranged to mechanically expand in its activated state to secure the garment to the respective body part of the subject or to provide force feedback, and an activation unit arranged to transmit said non-mechanical stimulus to each SMA individually in response to a defined activation sequence.

According to a seventh aspect, a garment assembly for facilitating or resisting movement of a subject, such as a person, animal, or inanimate object, wearing the garment assembly is provided. The garment assembly comprises a garment having a first anchor zone, a second anchor zone, each anchor zone acting to secure the garment to a respective body part of the subject, in use, at least one first smart material actuator(s) (SMAs) arranged between the first anchor zone and second anchor zone, and oriented along a first direction of the garment, and forming a first group of SMAs, at least one second smart material actuator(s) (SMAs) arranged between the first anchor zone and second anchor zone, and oriented along a second direction of the garment, and forming a second group of SMAs, wherein the first direction is different from the second direction, a force translation mechanism connecting the first anchor zone to the second anchor zone and comprising the first group of SMAs, and second group of SMAs, wherein each SMA is arranged to operate in an idle state, and an activated state triggered in response to a non-mechanical stimulus that causes the associated SMA to mechanically contract, and an activation unit arranged to transmit said non-mechanical stimulus to each SMA individually in response to a defined activation sequence.

According to an eight aspect, a garment assembly for facilitating or resisting movement of a subject, such as a person, animal, or inanimate object, wearing the garment assembly is provided. The garment assembly comprises a garment having a first anchor zone, a second anchor zone, each anchor zone acting to secure the garment to a respective body part of the subject, in use, at least one first smart material actuator(s) (SMAs) arranged between the first anchor zone and second anchor zone, and oriented along a first direction of the garment, and forming a first group of SMAs, at least one second smart material actuator(s) (SMAs) arranged between the first anchor zone and second anchor zone, and oriented along a second direction of the garment, and forming a second group of SMAs, wherein the first direction is different from the second direction, a force translation mechanism connecting the first anchor zone to the second anchor zone and comprising the first group of SMAs, and second group of SMAs, wherein each SMA is arranged to operate in an idle state, and an activated state triggered in response to a non-mechanical stimulus that causes the associated SMA to mechanically expand, and an activation unit arranged to transmit said non-mechanical stimulus to each SMA individually in response to a defined activation sequence.

According to a ninth aspect, a garment assembly for facilitating or resisting movement of a subject, such as a person, animal, or inanimate object, wearing the garment assembly is provided. The garment assembly comprises a garment. The garment assembly further comprises an actuator assembly comprising a first anchor zone, a second anchor zone, each anchor zone acting to secure the garment to a respective body part of the subject, in use, at least one smart material actuators (SMAs) connected between the first anchor zone and second anchor zone, and forming a first group of SMAs, and a force translation mechanism connecting the first anchor zone to the second anchor zone and comprising the first group of SMAs. Each SMA is arranged to operate in an idle or deactivated state, and an activated state triggered by a non-mechanical stimulus that causes a geometrical change in the associated SMA. The garment assembly further comprises an activation unit arranged to transmit said non-mechanical stimulus to each SMA individually in response to a defined activation sequence.

### BRIEF DESCRIPTION OF THE DRAWINGS

A number of examples will now be shown, by way of example, with reference to the following drawings, in which:

FIG. 1 illustrates a top view of a garment assembly according to an example;

FIG. 2 illustrates a top view of a garment assembly with an alternative force translation mechanism configuration according to an example;

FIG. 3 illustrates a top view of a garment assembly with a further alternative force translation mechanism configuration according to an example;

FIG. 4 illustrates a top view of a garment assembly with a yet a further alternative force translation mechanism configuration according to an example;

FIG. 5 illustrates a top view of a garment assembly with yet another alternative force translation mechanism configuration according to an example;

FIG. 6 illustrates a top view of a photo-responsive smart actuator 1D array according to an example;

FIGS. 7a and 7b illustrates a top view of a group of SMAs connected in sequence in an idle state and activated state, respectively;

FIG. 8 shows a side view of part of a garment assembly in an idle state (top half), and activated state (bottom half);

FIG. 9 shows a top view of a garment assembly comprising two groups of in-sequence connected SMAs laterally detached and running in parallel;

FIG. 10 shows a top view of a garment assembly comprising two groups of in-sequence connected SMAs laterally detached and extending in different directions in relation to the first and second anchor zones;

FIG. 11 shows a top view of a garment assembly comprising three groups of in- sequence connected SMAs that are laterally detached, where two of the groups run in parallel to one another, while another group of SMAs extends in a different direction;

FIG. 12 shows a top view of a garment assembly according to a further example wherein each anchor zone comprises one or more SMAs;

FIG. 13 shows a side view of a garment assembly having five groups of laterally detached in-sequence connected SMAs according to another example;

FIG. 14 shows a side view of the garment assembly of FIG. 13 wherein the activation unit comprises a number of light strips corresponding to each of the groups of in-sequence connected SMAs;

FIG. 15 shows a side view of the garment assembly of FIG. 13 wherein the activation unit comprises LED panel covering the groups of in-sequence connected SMAs; and

FIG. 16 shows a side view of the garment assembly of FIG. 13 wherein the activation unit comprises a luminescent panel.

### DETAILED DESCRIPTION

A general idea of the present invention is to provide a garment assembly having a garment, and one or more discrete smart material actuator(s) configured to undergo a change in its physical material properties. The change in physical material properties may result in a geometrical change. The geometrical change may refer to a change in the geometrical extension, such as the length or width, of the associated SMA. The geometrical change may relate to the associated SMA mechanically contracting or mechanically expanding, upon receiving the non-mechanical stimulation. The associated change in physical material properties leading to a geometrical change may create or result in a force that is translated to one or more anchor zones of the garment. The physical material properties may relate to stiffness (relating to the ability of the material to withstand stress without breaking the material or plastically deforming or permanently deforming the material) or strength of the material. The anchor zones are arranged to provide a friction or snug fit against a body part of the subject wearing the garment or garment assembly. The force created by the activation of the smart material actuator(s) is translated to the body part of the subject via the anchor zone(s) of the garment using a force translation mechanism. Depending on the type of application the created force may act to resist a movement of the associated body part of the subject or facilitating a desired movement of the subject. The force created may in some examples be varied in magnitude by varying the magnitude of the non-mechanical stimulation, or by varying other parameters associated with the non-mechanical stimulation, such as light-polarisation angle, etc.

In some examples, upon mechanically contracting the stiffness of the associated SMA material is increased. In some examples, upon mechanically expanding the stiffness of the associated SMA material is decreased.

In some examples, the garment assembly may be used as a wearable workout device, where the user acts to work against the force created upon activation of the associated smart material actuators.

In other examples, the garment assembly may be used as a wearable limb movement facilitation device, allowing users with impaired mobility to achieve a higher degree of mobility.

In other examples, the garment assembly may be used as medical rehabilitation device, allowing patients with impaired mobility to achieve a higher degree of mobility.

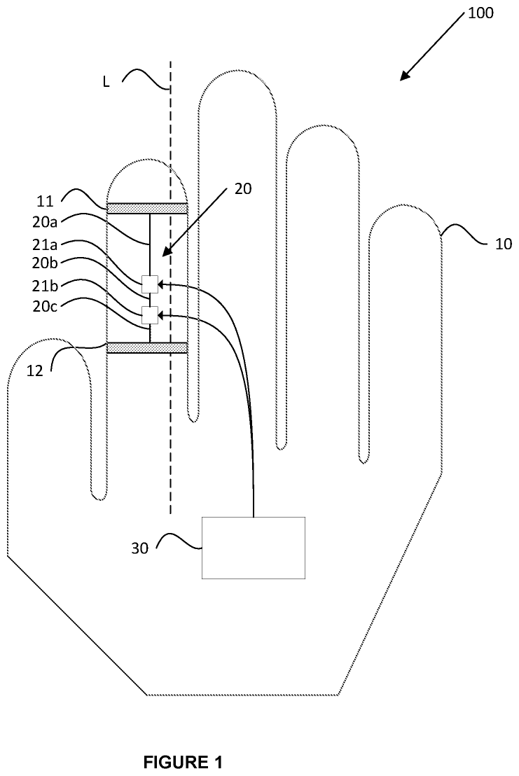

With reference to FIG. 1 a first example of a garment assembly 100 for facilitating or resisting movement of a subject, such as a person, wearing the garment assembly is shown. The garment assembly 100 comprises a garment 10, depicted as a glove in FIG. 1, having a first anchor zone 11, a second anchor zone 12. Each anchor zone 11, 12 acts to secure the garment 10 to a respective body part of the subject, in use. The garment assembly further comprises at least two first smart material actuators (SMAs) 21a, 21b connected in sequence between the first anchor zone 11 and second anchor zone 12. The at least two SMAs connected in sequence form a first group of SMAs. The garment assembly 100 further comprises a force translation mechanism 20 connecting the first anchor zone 11 to the second anchor zone 12 and comprising the first group of SMAs 21a, 21b. Each SMA is arranged to operate in an idle state or non-activated state, and in an activated state triggered by a non-mechanical stimulus causing the associated SMA to mechanically contract. Further, the garment assembly 100 comprises an activation unit 30 arranged to individually transmit said non-mechanical stimulus to each SMA in response to a defined activation sequence.

The anchor zones disclosed herein are arranged to be secured to the respective body part of the subject. When secured the respective anchor zone provides a snug fit against a body part of the subject. Further the anchor zone is arranged so that when secured to the associated body part, it will be unable to move in relation to the body part to which it is attached. In particular, the anchor zone is arranged to provide friction between the garment and the body part. The friction provided are designed to exceed the force created by the SMAs connected thereto upon activation and deactivation. Hence, when the SMAs are activated, the resulting force created is less than a force required to enable any movement of the secured anchor zones in relation to the body part to which they are attached. Expressed differently, the force required to enable movement of the anchor zone in relation to the associated body part once secured is designed to be larger than the force created by the associated SMAs when activated and/or deactivated. In some configurations, to maximise the friction between the garment (or anchor zones) and the body part to which the anchor zones are attached, the anchor zones are arranged to surround the associated body part. Hence, the anchor zones may be formed as a sleeve or the like. In some configurations, the portion of the garment being in contact with the body portion of the subject at the location of the anchor zone may be provided with a material, providing for high friction against the body part of the subject.

As will be explained further herein, in some examples the associated SMA may also be configured to mechanically expand in its activated state.

Common for all SMAs disclosed herein, in the activated state, the non-mechanical stimulus will cause a change in physical material properties. The change in physical material properties may result in a geometrical change in the SMA as compared to the geometrical extension of the SMA in its associated deactivated or idle state. Further, following the activated state, in the deactivated state that is triggered by the removal of the non-mechanical stimulus, the associated SMA will undergo another change in its physical material properties, resulting in a geometrical change for the SMA to return towards its geometrical extension in the deactivated or idle state. As such, the geometrical change, such as the change in geometrical extension of the SMA, may be associated with the SMA mechanically contracting or mechanically extending. It also follows that if the SMA is configured to mechanically contract upon receiving a particular non-mechanical stimulus, it will mechanically expand upon the removal of said non-mechanical stimulus. The opposite also holds true. Hence, if the SMA is configured to mechanically extend upon receiving a particular non-mechanical stimulus, it will tend to mechanically contract upon the removal of said non-mechanical stimulus.

Rather than utilizing a long SMA, e.g., an SMA fibre or yarn extending completely between the two anchor zones, utilizing discrete SMAs connected in sequence between the two anchor zones, a desired overall total force may be attained while at the same time utilizing the higher response rate of the discrete SMAs. Connecting the SMAs in sequence means that the SMAS are connected one after another, such as in series, between the first anchor zone and second anchor zone. Hence, the term “in sequence” implies that two or more SMAs are connected in series after one another between the first anchor zone and second anchor zone. One or more linkages may be arranged to attach the respective SMAs together as will be further discussed herein.

Further, arranging two or more SMAs in sequence allows for activation of a number of the SMAs in the sequence in a local region between the two anchor zones. When combined with activation of a second group of neighbouring in sequence SMAs in a second local region, this allows for an overall torsional force (still with a longitudinal component) to be created. This torsional force makes the garment more versatile and allows for the garment assembly to trigger, resist, or facilitate also torsional movements of the user wearing the garment assembly, in use.

The first group of SMAs may be connected in sequence along a first direction, such as a longitudinal direction, between first anchor zone and the second anchor zone.

A technical effect of the force translation mechanism is to translate a force, such a pulling force, that is created by each activated SMA, between the respective activated SMA and the anchor zones 11 and 12. As the at least two SMAs are connected in sequence between the first anchor zone 11 and second anchor zone 12, upon activation of the associated SMAs, the force translation mechanism will act to pull or force the first anchor zone towards the second anchor zone and vice versa. Accordingly, in response to the activation the created force acts to reduce the longitudinal distance between the first anchor zone 11 and the second anchor zone 12 is reduced as compared to the idle or deactivated state.

In situations where there is no external force acting in an opposite longitudinal direction to that of the created force and being equal or larger than the created force, the longitudinal distance between the two anchor zones will be reduced.

For SMAs configured to mechanically expand in its activated state, a technical effect of the force translation mechanism is to translate a force that is created by each activated SMA, between the respective activated SMA and the anchor zones 11 and 12. As the at least two SMAs are connected in sequence between the first anchor zone 11 and second anchor zone 12, upon activation of the associated SMAs, the force translation mechanism will act to reduce any pulling force between the first anchor zone and the second anchor zone. Alternatively, upon activation the mechanical expansion of the SMA may result in an increase in the longitudinal distance between the first anchor zone and second anchor zone. The mechanical expansion may be said to relax the SMA and making it less stiff in some examples. Accordingly, in response to the activation the created force acts to increase the longitudinal distance between the first anchor zone 11 and the second anchor zone 12 as compared to the idle or deactivated state.

When an external force, acting against the created force, is equal to the created force, the longitudinal distance will remain constant.

When the created force has a vector component acting along the direction of the external force, the created force acts to facilitate the associated external movement caused by the external force. In this example, the created force adds to the external force. In this situation the external force may cause a greater change to the longitudinal distance between the first anchor zone and the second anchor zone as compared to the change caused by the force created by the activated SMAs alone. Hence, if the change in the longitudinal distance caused by the created force relates to a decrease (e.g. due to mechanical contraction), then the external force may allow for a resulting even greater decrease (i.e. larger decrease) in the longitudinal distance. Conversely, if the change in the longitudinal distance caused by the created force relates to an increase (e.g. due to mechanical expansion), then the external force may allow for a resulting even greater increase (i.e. larger increase) in the longitudinal distance.

When the created force has a vector component acting against the direction of the external force, the created force acts to resist the associated external movement caused by the external force. In this situation the external force may cause a reduced change to the longitudinal distance between the first anchor zone and the second anchor zone as compared to the change caused by the force created by the activated SMAs alone. In this situation, if the change in the longitudinal distance caused by the created force relates to a decrease (e.g. due to mechanical contraction), then the external force may allow for a resulting reduced decrease (i.e. smaller decrease) in the longitudinal distance. Conversely, if the change in the longitudinal distance caused by the created force relates to an increase (e.g. due to mechanical expansion), then the external force may allow for a resulting reduced increase (i.e. smaller increase) in the longitudinal distance.

In some situations, when the external force is larger than the created force, the longitudinal distance may be increased.

In some examples, the associated SMAs are controlled to create a force depending on the magnitude of the external force, or on a sensed movement of the garment associated by the external force.

As will be further elucidated below, in some examples the magnitude of the force created by activation of the associated SMAs is tailored to continuously match that of the external load, to so that the longitudinal distance between the first anchor zone 11 and the second anchor zone 12 is maintained constant or substantially constant. This allows for providing force feedback to the user wearing the garment or garment assembly. In this way, the garment may be used to cancel out a force or stop a motion of a limb secured to a first anchor zone and/or second anchor zone, in use. Hence, the associated SMAs may be controlled to create a force to match that of an external force to maintain the longitudinal distance between the first and second anchor zones constant or substantially constant.

The external force may be associated with a force resulting from the user of the garment moving a limb or body part to which the garment is secured.

The anchor zones 11 and 12 may be arranged to secure the garment to specific body parts of the subject wearing the garment or garment assembly. For example, when the anchor zones are secured to the body of the subject wearing the garment at either side of a joint, such a finger joint, activation of the associated SMAs will act to bend or straighten the associated limb around the joint, depending on the arrangement of the force translation mechanism.

The defined activation sequence may be associated with a desired movement and/or orientation of a body part of the subject wearing the garment or garment assembly. In some examples, the defined activation sequence may be selected and/or controlled by the user. A controller (not shown), operatively coupled to the activation unit 30, may execute the defined activation sequence in response to input from an operator or the user wearing the garment or garment assembly.

The controller may be a microcontroller comprising a processor operatively coupled to a memory and arranged to execute a number of computer executable instructions, optionally stored on a non-transitory computer readable medium. The microcontroller may form part of an integrated circuit comprising a memory, and input/output peripherals. The microcontroller operates by executing a program stored in its memory, which controls various functions and operations. Several types of microcontrollers may be used, each with its own set of features, capabilities, and applications. For example, some microcontrollers are designed for low power consumption which makes them ideal for battery-operated devices, while others are optimised for high performance making them suitable for demanding applications. For the purpose of the present disclosure the controller may be any available microcontroller suitable for processing sensor information from any sensors of the garment assembly, and for controlling the operation of the activation unit according to set instructions.

The processor may be operatively coupled to a control circuit for controlling the operation of the activation unit 30. In some examples, the control circuit forms part of the activation unit 30.

The control circuit may be arranged to control stimulation intensity of the associated activation unit 30. Depending on the type of activation unit and associated SMA, the stimulation intensity may relate to light for photo responsive SMAs, voltage level for Dielectric or electro restrictive elastomer actuators (DEA), Conductive polymer actuators (CP), or Electroactive polymer actuators (EAP) or magnetic flux for Magneto strictive actuators (MA).

In some examples, the activation sequence may be derived by the controller based on sensor information accessed by the controller. For example, the sensor information may comprise strain sensor information from one or more strain sensors sensing a degree of bending of the garment. Alternatively, or additionally the sensor information may comprise accelerometer or gyroscopic information from one or more accelerometers or gyroscopes attached to the garment.

Optionally, the sensor information may comprise Electroencephalogram (EEG) or Electromyography (EMG) information.

Based on the sensor information, the controller may be configured to control (e.g. adjust or regulate), either intensity of stimulus, percentage of SMA material stimulated, or a ratio of stimulated SMA material and varied directions to create a desired created force or longitudinal distance displacement between the first anchor zone and second anchor zone.

In some examples, the sensor technology used may utilize base stations (that can be placed around a room to spatially sense full body movements and the sensed data may then be accessed by to the control unit, e.g. via Bluetooth, to control the SMAs), ultrasonic vibration sensors, VR headset tracking cameras, strain sensors on the glove and AI (Artificial Intelligence) motion tracking and motion capture technologies.

In some examples, the garment 10 comprises a fabric to which the first anchor zone and the second anchor zone is fixedly attached. At least one of the respective anchor zones may be fixedly attached to the fabric via a suitable attachment, such as using bolts, rivets, seams, Velcro, or any other known attachment suitable.

In some applications at least one anchor zone may form part of a finger cap of the garment assembly, wherein the finger cap secures to a respective fingertip of the subject.

In some examples the respective anchor zone may be arranged at a predetermined location in the garment, wherein said location corresponds to the location of a body joint of the subject when the garment is worn by the subject.

The fabric may also be fixedly or releasably attached to one or more of the SMAs.

In some examples, the fabric may be made of any conventional garment material, such as but not limited to cotton, polyester, wool, nylon, elastane and/or a breathable garment fabric.

In some examples, the garment forms an exoskeleton or exosuit.

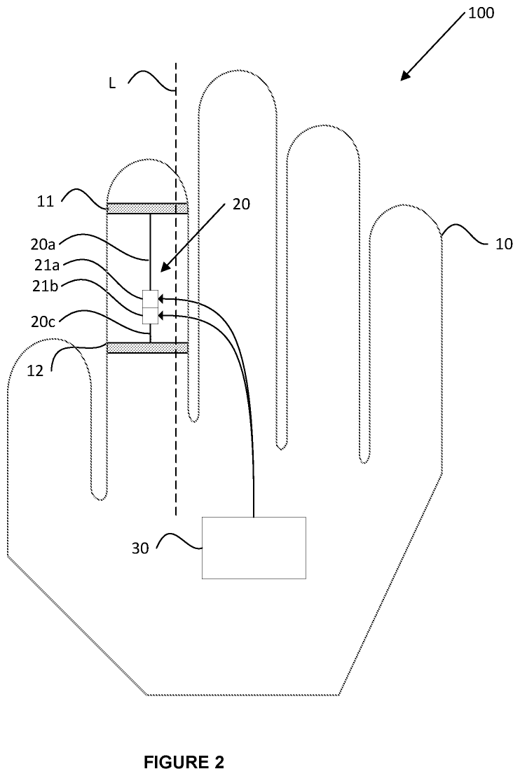

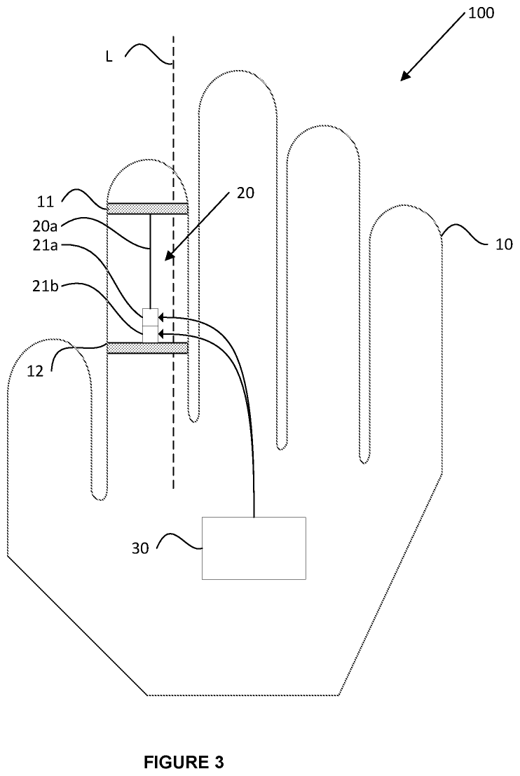

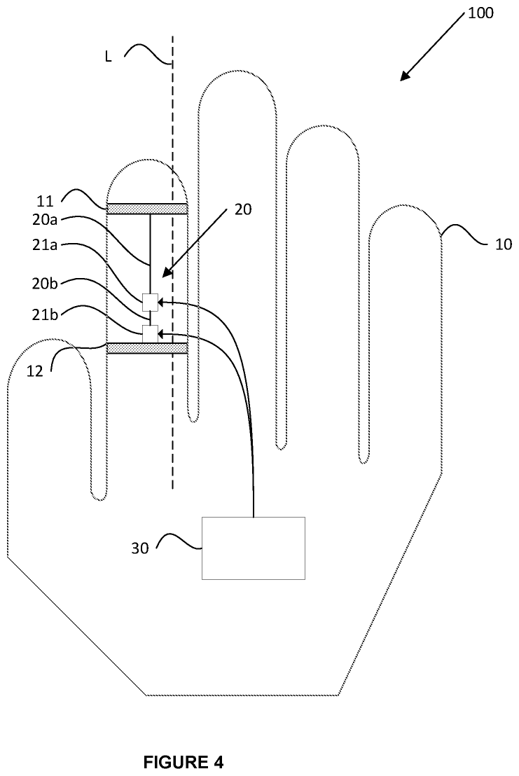

In some examples, as shown with reference to FIG. 2, each SMA in the sequence is directly attached to one or more SMAs. Alternatively, or additionally each SMA in the sequence could be directly attached to one or more anchor zone(s), as shown with reference to FIGS. 3 and 4. Each SMA may be directly attached to one of the further SMA(s) or anchor zone(s) at either a first longitudinal end 21a′, 21b′ or second longitudinal end 21a″, 21b″ thereof.

In some examples, each SMA is directly attached to next SMA in sequence and/or to an associated anchor zone 11 and 12 using an adhesive, seam, clip, zip, clasp, hook, or the like.

However, in alternative examples, at least one of the SMAs may be attached to a neighbouring SMA or anchor zone via a linkage, schematically shown as solid lines linking the SMAs to the respective anchor zones in FIGS. 1 to 4, that in turn is directly attached to said SMA and the neighbouring SMA or anchor zone. The linkage member(s) form part of the force translation mechanism 20.

The number of linkages used may e.g. depend on the physical distance between the two connected anchor zones in the idle state, and the number of and associated size of the SMAs connected in sequence therebetween. The number of SMAs in turn may be selected based on the desired magnitude of the force to be created between the anchor zones when each SMA is activated. For example, three linkages are shown with reference to FIG. 1, two linkages are shown with reference to FIGS. 2 and 3b, whereas one linkage is shown with reference to FIG. 3a.

The linkages of the force translation mechanism may extend along or parallel to a longitudinal direction or axis (L) between the first and second anchor zone, along which longitudinal direction the respective SMAs are connected in sequence. To this end, the linkages of the force translation mechanism may be referred to as longitudinal linkages.

For example, considering a garment assembly having two connected anchor zones displaced 100 mm apart in an idle state along a longitudinal direction or axis, and with four SMAS, each 20 mm in size, a resulting space of 20 mm, i.e. 100 mm−4*20 mm=20 mm out of the 100 mm may be provided with one or more longitudinal linkages.

The force translation mechanism is formed by the associated SMAs, and their associated attachments to other SMAs or anchor zones, and any optional linkages.

An underlying idea of the force translation mechanism is to provide minimal slack for each group of in sequence SMAs connected between the associated anchor zones. In this way, a majority of the force created when activating the SMA, e.g. up to 95%, may be translated from the associated SMAs to the anchor zones. In some examples, the force translation mechanism has a force translation efficiency of about 85% to about 95%. While a 100% force translation efficiency would be ideal, in practice there will always be efficiency losses caused by undesired deformation of the force translation mechanism, or adjacent materials connected thereto, resulting in reducing the overall force translation efficiency from the ideal value.

In some examples, the one or more linkages may comprise comprises at least one of a fibre, yarn, cable, or wire, with an elastic modulus δ=stress/strain, e.g. a Youngs modulus, being higher than a predetermined threshold.

The predetermined threshold may be set higher than the force created by the associated SMAs, thereby limiting the tendency of the linkage to deform along the longitudinal axis along which the SMAs are connected in sequence between first anchor zone 11 and second anchor zones 12, when the SMAs are activated.

In an example, one or more linkages may be made of a material that at least to an extent is rigid, non-flexible, or inelastic along a longitudinal direction thereof. This allows the associated linkages to translate the force created along or parallel to said longitudinal direction when the associated SMAs are activated and/or deactivated. However, it should be appreciated that the one or more linkages may be made of a material having a degree of compliance or damping characteristics. The compliance or damping characteristics may be utilized to smoothen out abrupt movements resulting from the activation/deactivation of the neighbouring SMAs. Hence, while rigidity, non-flexibility, and/or inelasticity may be considered the dominating material characteristics allowing the respective linkage member efficiently translating the forces created by the connected SMAs contracting or expanding, it should be appreciated that the material does not have to be perfectly, fully, or otherwise 100% rigid, non-flexible, or inelastic. However, it should be noted that increasing the compliance too much would have a detrimental effect on the force translation capabilities of the linkage member. The linkages may be strong enough to withstand failure at maximum compounded force generation, e.g. at 4.5 kg per finger. On the other hand, the linkages may have a degree of flexibility along any non-longitudinal direction. For example, a cable like linkage may be preferred over a push rod type linkage for this reason.

The linkages may have a first end and second end opposite the first end. The respective ends of the linkage may be arranged to be anchored to either an anchor zone 11, 12 or to a longitudinal end an SMA of the sequence of SMAs forming a group of the SMAs. In some examples, the linkages are anchored using an adhesive or mechanical attachment, such as a seam.

In some examples, a shown with reference to FIGS. 1 to 4, the force translation mechanism 20 comprises at least one of: a first linkage 20a connected between and attached to the first anchor zone and an SMA closest in sequence to the first anchor zone 11, a second linkage 20b connected between and attached to two closest neighbouring SMAs, and a third linkage 20c connected between and attached to the second anchor zone and a SMA closest in sequence to the second anchor zone.

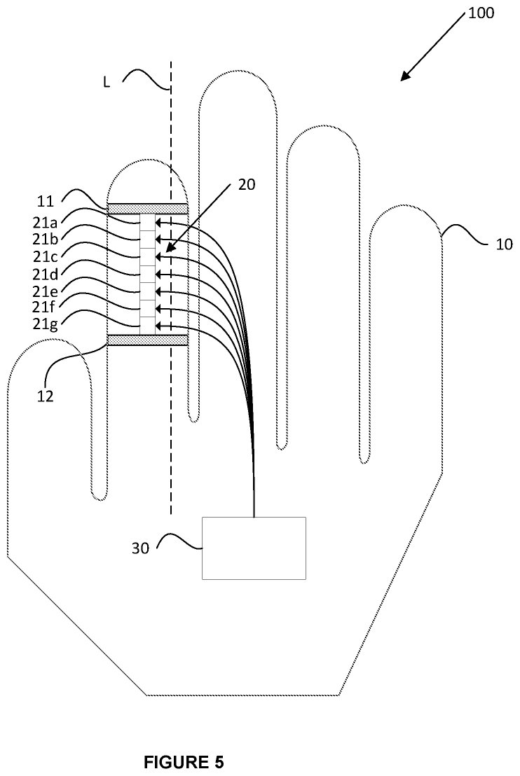

In some examples, as shown with reference to FIG. 5, the force translation mechanism may comprise no linkages, whereby each SMA 21a to 21g of the sequence is directly attached to either another SMA and anchor zone or two neighbouring SMAs.

In an alternative example, the force translation mechanism may further be connected to a damper arranged to dampen out the reduction in longitudinal distance between the associated anchor zones 11, 12 upon activation of the associated SMAs 21a-21g. The damper may comprise a tension spring (not shown), or an elastic band or the like, that smoothens out any abrupt relative movements between the first 11 and second 12 anchor zones. This in turn allows for the associated body parts secured to the respective anchor zones to move smoothly upon activation of the associated SMAs. For example, a damper may be attached between the first anchor zone and the first SMA in sequence. Hence, each group of SMAs may be connected to damper arranged to dampen out the force created upon activation of the associated SMAs, thereby smoothing out any abrupt reduction of the longitudinal distance.

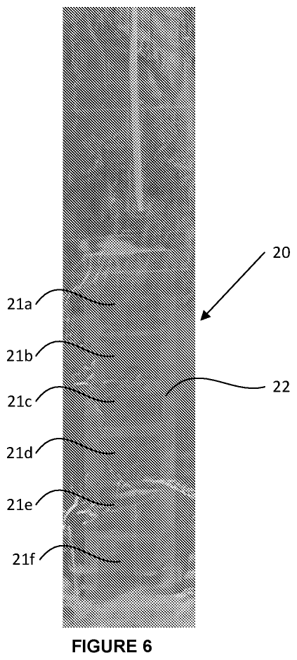

With reference to FIGS. 5 and 6 the first group of in-sequence SMAs 21a-21g may be formed as a strip or 1D array of discrete SMAs. In some examples, the strip or array may comprise a film 22, e.g. a thin film, encapsulating the SMAs. In this way, the film forms one or more linkages of the force translation mechanism. The film may be selected from a material configured to allow for conforming with the curvature of the human body.

For example, the thin film may comprise a coating, such as a clear coating, or plastic film applied over the array of SMAs, wherein the clear coating or plastic film is arranged to translate actuation forces through its structure.

In some examples, the thin film may be coated to act as a light guide to allow for light to be contained within the thin film. This is particularly advantageous when the SMA is a photo-responsive material that is activated by light. Using the thin film as a light guide, while also acting as a linkage member of the force translation mechanism allows for a light-weight solution.

In some examples the film may have a thickness in the range of 200 nm to 1 mm.

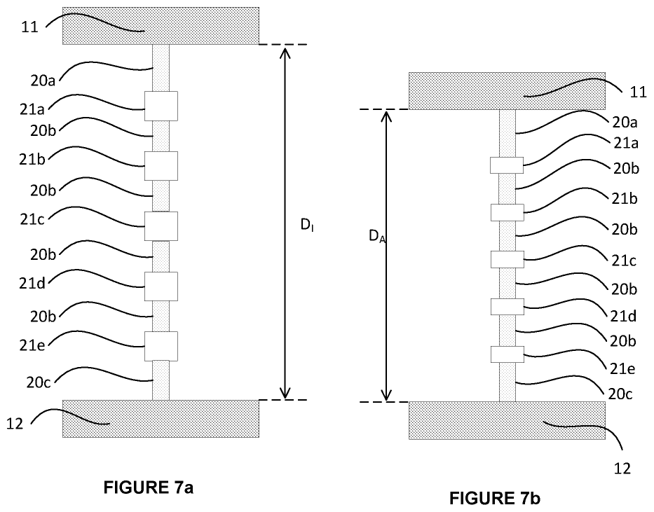

FIG. 7a shows an example where a first group of SMAs 21a-21e connected in sequence are present in their idle state, i.e. non-activated state. In the idle state the longitudinal distance between the first anchor zone 11 and the second anchor zone 12 is represented by DI. In the example the respective SMA is configured to mechanically contract upon activation. Upon activation by the activation unit (not shown) the respective SMA 21a-21e mechanically contracts and this situation is shown in FIG. 7b. Upon no external force, or an external force being less than that created by the activation of the associated SMAs, as the SMAs 21a-21e are physically linked to the anchor zones, by the force translation mechanism 20 and in this example via a number of linkages 20a-20c of the force translation mechanism, the created forced resulting from the mechanical contraction, is translated to the respective anchor zones. This is turn acts to pull the first anchor zone 11 and the second anchor zone 12 towards each other, whereby the longitudinal distance between the anchor zones in the activated state is reduced in comparison to that of the idle state. The resulting reduced longitudinal distance between the first anchor zone 11 and the second anchor zone 12 in the activated state is represented by D_{A} in FIG. 7b. It may be observed from FIGS. 7a and 7b that the longitudinal distance D_{A} clearly is smaller than the longitudinal distance D_{I}. However, upon the presence of an external force, such as an external force acting in an opposite direction to that of the created force, and having a magnitude equal to that of the created force, the longitudinal distance may be kept constant or substantially constant, as discussed above.

In an alternative example, where the respective SMA is configured to mechanically extend upon activation, the longitudinal distance between the first anchor zone 11 and the second anchor zone 12 is increased upon activation. In such an example, FIG. 7b may refer to the idle, non-activated state, whereas FIG. 7a represents the activated state.

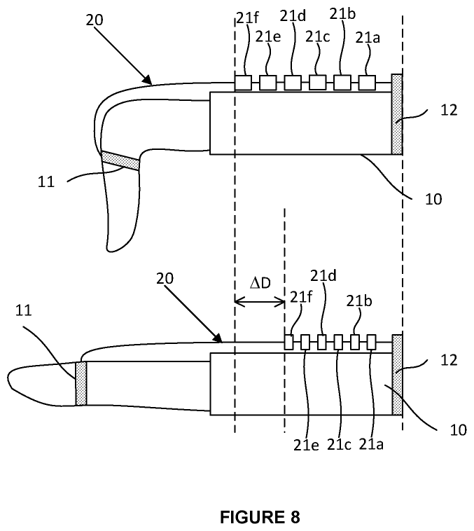

FIG. 8 shows a side view of part of the garment assembly 100 according to an example. In this example the respective SMAs are configured to mechanically contract upon activation. Here, the garment is a finger glove, where a first anchor zone is arranged in one of the ends of a finger sleeve of the glove. The location of the first anchor zone 11 in this example is that corresponding to a location just on the fingertip side of the proximal interphalangeal joint (PIP) of the finger of the subject wearing the garment or garment assembly 10, in use. A second anchor zone 12 is arranged at another end of the garment 10, e.g. around the wrist of the subject wearing the garment or garment assembly. A number of SMAs 21a-21f are arranged in sequence between the first anchor zone 11 and the second anchor zone 12. The SMAs are linked via a number of linkages. At least one of the linkages, e.g. that between the first anchor zone 11 and SMA 21f, comprise a wire acting as a force translating wire. The force translating wire has a fix length and has a tensile strength higher than the combined force produced by the SMAs when activated. As such, the length of the force translating wire will remain constant or substantially constant upon activation of the associated SMAs 21a-21f. In the top half of FIG. 8, the associated SMAs are in their respective idle states. In this situation the finger sleeve of the garment 20 is in a bent configuration. The lower half of FIG. 8 represent a situation where the SMAs have been activated by the activation unit 30 (not shown). Upon activation the SMAS mechanically contract, which results in a shortening the length of the force translation mechanism and the distance between the first and second anchor point by ΔD=D_{I}-D_{A} (compare with FIGS. 7a and 7B). Hence, while the individual lengths of the linkages remain the same upon activation, the reduction in length of the force translation mechanism is fully due to the mechanical contraction of the SMAs. The mechanical contraction results in a force pulling the first anchor zone 11 towards the second anchor zone 12 or vice versa. In use, when the garment is worn by a subject, as the tip of the finger to which the first anchor zone 11 is secures has a lower mass beyond the first anchor zone towards the tip of the glove than that of the subject's body beyond the second anchor zone, the first anchor zone 11 will move towards the second anchor zone 12 as shown in FIG. 8.

In an alternative example, where the respective SMA is configured to mechanically extend upon activation, the longitudinal distance between the first anchor zone 11 and the second anchor zone 12 is increased upon activation. In such an example, FIG. 8b may refer to the idle, non-activated state, whereas FIG. 8a represents the activated state.

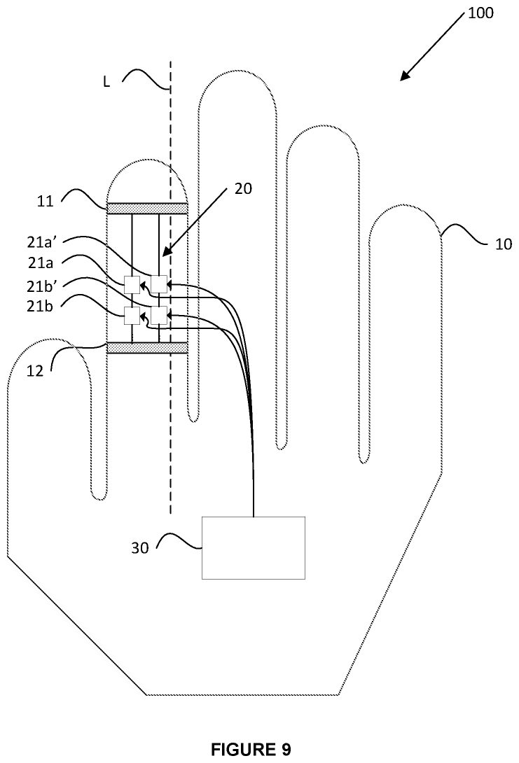

With reference to FIG. 9, in order to create an increased force between the first anchor zone 11 and second anchor zone 12, a second group of in-sequence SMAs 21a′, 21b′, optionally formed as a second strip or second 1D array, may be connected to the first anchor zone 11 and second anchor zone 12 and spaced laterally to that of the first group of SMAs.

In some examples, the second group of in-sequence SMAs, may be arranged in parallel to the first group of SMAs. Such an example is shown with reference to FIG. 9.

The second group of in-sequence SMAs may be laterally detached from the first group. This implies that no part of the second group is attached to the first group other than indirectly via the first and second anchor zones. Laterally detaching the first group from the second group allows for the created force from each group of SMAs to substantially only act along the longitudinal direction along which the in-sequence SMAs are connected.

The at least two laterally detached groups of in-sequence SMAs may be said to form a laterally detached 2D array of in sequence SMAs. The lateral linkages may also comprise at least one SMAs configured to adjust the lateral distance (such as reducing by mechanically contracting or increasing by mechanically expanding upon activation or deactivation) between the two laterally detached groups of SMAs.

In an alternative example, two or more groups of SMAs may be laterally attached to one another via lateral linkages to form a 2D array. The lateral linkages may allow the groups of SMAs to maintain a desired lateral spacing when activated in parallel. In this way, the lateral attachments provide for lateral stabilisation of the two groups when activated. This is particularly advantageous when the two or more groups are designed to be activated in parallel, as this allows for minimized skewing or splaying of neighbouring SMAs as compared to when each group is activated while neighbouring groups are operating in their idle states.

The at least two laterally attached groups of in-sequence SMAs may be said to form a laterally attached 2D array of in sequence SMAs.

Hence, depending on the application, the first group of SMAs 21a-21g and the one or more second groups of SMAs 21a′-21g′ may be arranged in an array with groups of SMAs that are either laterally detached or laterally attached to another group of SMAs of the array.

In some examples, each SMA group of the array is arranged in parallel with the other SMA groups, whereby the respective SMAs of each group is connected in-sequence along the same direction, such as a longitudinal direction (L) extending between the first anchor zone 11 and the second anchor zone 12.

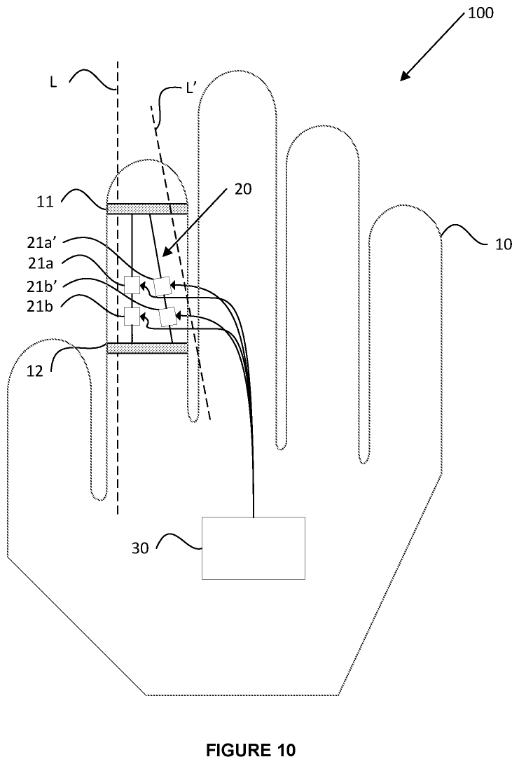

In some examples, the associated SMAs of the second each second group of SMAs 21a′-21g′, that are connected in sequence between the first anchor zone 11 and second anchor zone 12 may be connected in sequence along a second direction (L′) between first anchor zone and the second anchor zone. The second direction of the second group of SMAs may be different to the first direction of the first group of SMAs, as shown with reference to FIG. 10.

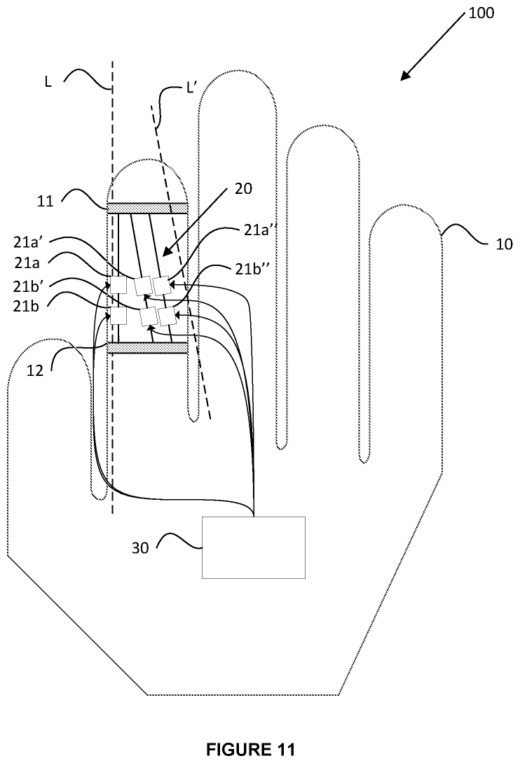

In some examples, with reference to FIG. 11, the second group of SMAs 21a′-21g′ are formed in a laterally detached or laterally attached array comprising at least one further second group of in-sequence connected SMAs 21a″-21g″ arranged in parallel to the second direction (L′), and the first group of SMAs 21a-21g is arranged along a first direction (L), wherein the first direction (L) differs from the second direction (L′).

In some examples, a combination of laterally detached and laterally attached SMA arrays may be used.

In some examples, one or more first groups of SMAs is arranged in a first layer of the garment, and one or more second groups of SMAs are arranged in a second layer of the garment, wherein the second layer at least partly overlaps the first layer.

The SMAs of each layer may be arranged in sequence along a set direction, which may differ from the direction along which in sequence SMAs of a neighbouring layer are arranged. Arranging the SMAs is layers may increase the density of SMAs, thereby enabling creation of a larger force when the more SMAs are activated together. Further, when by arranging the SMAs in layers, where each layer comprises SMAs connected in sequence along a direction that differs from the direction along which the in sequence SMAs of a neighbouring layer are arranged, it is possible to create a torsional force that may assist in rotation of the associated body part or limb of the subject wearing the garment or garment assembly. Hence, upon activation of the SMAs of each layer with SMAs connected in sequence along different directions, the associated body part or limb will be pulled in the different directions simultaneously, thereby creating a both a force having a longitudinal component and a force having a lateral component, resulting in an overall torsional force.

In some examples, the respective anchor zone 11, 12 may be arranged to surround a body part of the subject in use. For example, the anchor zones may form a collar or sleeve arranged to be secured to an associated body part or limb of the subject wearing the garment or garment assembly, in use.

In some examples the collar may be formed by a stretchable fabric arranged to provide a friction fit with the associated body part when the body part or limb is inserted into the anchor zone. comm

At least one anchor zone may be arranged in a sleeve of the garment, such as shown with reference to FIGS. 4a and 4b, that surrounds a body part or limb of the subject wearing the garment or garment assembly, in use.

The anchor zones may be secured to the subject e.g. using elastic garters, belt-ratchet type mechanisms (similar to those used of bag strap adjustment), Velcro in conjunction with elastic strips, buttons, grippy silicones and/or zips. The anchor zones or their respective attachment points to the garment may in some alternatives be modular, such as interchangeable, thereby allowing for swapping out attachment points or types (such as replacing a Velcro attachment with a hook attachment) in a modular way to fit the particular design. This allows the garment or garment assembly to enable for a wide range of attachment types including but not limited to: Velcro, laces or strings, screws, nuts and bolts, buckles, hooks etc.

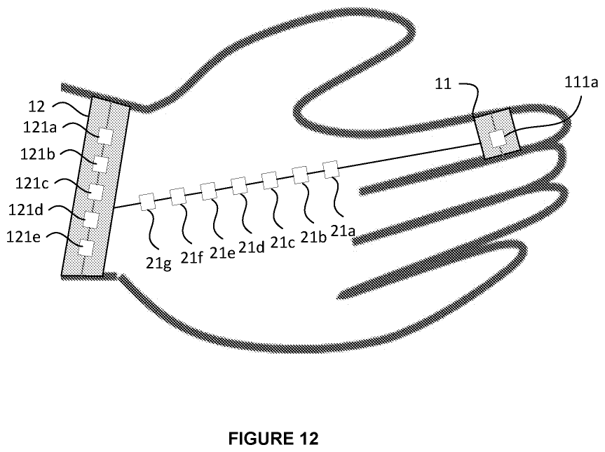

In some examples, the first anchor zone 11 and/or the second anchor zone 12 may further comprise at least one second SMA 111, 121, arranged to mechanically contract in its activated state to secure the garment to the respective body part of the subject or alternatively providing force feedback to the subject by creating a force acting against the external force exerted by the subject on the garment. FIG. 12 shows an example where the second anchor zone 12 comprises a number of in sequence SMAs 121a-121e and at least one SMA 111a arranged in the first anchor zone 11. When the anchor zone is arranged in a sleeve of the garment, this allows for the interior circumference of the sleeve to reduce by the force created when the associated SMAs are mechanically contracted in the absence of an external force exceeding the force created, thereby reducing the diameter of the sleeve in turn providing a secure fit against the associated body part or limb. When an external force is present, the force created by the SMAS may provide force feedback simulating tactile sensations such as pressure on the subject wearing the garment or garment assembly.

Alternatively, the respective SMA of the respective anchor zone may be configured to mechanically expand in its activated state. Upon activation this would allow the anchor zone to alleviate the force or pressure sensed by the subject wearing the garment or garment assembly to provide for a looser, i.e. less secure, fit. This may be particularly advantageous when the garment assembly while worn is not operated to trigger, facilitate or resisting movement, such as when in a standby mode. It should be appreciated that a combination of SMAs that mechanically contract upon non-mechanical stimulus and SMAs that mechanically expand upon activation may be utilized. In some examples the garment assembly comprises at least two SMAs, wherein at least one of the at least two SMAs is configured to mechanically contract upon activation, and at least one of the at least two SMAs is configured to mechanically expand upon activation. In some examples, the at least two SMAs may be arranged in sequence between a first and second anchor zone. In some examples, the at least two SMAs may be arranged in sequence in an anchor zone. Alternatively, the at least two SMAs may be separately provided (i.e. not is sequence) in the garment assembly.

It should be appreciated that the garment assembly may according to some examples utilize a combination of different types of SMAs, such as those explained in further detail below. For example, a combination of dielectric SMAs and photo responsive SMA may be used.

Further, in some examples the garment assembly may comprise two or more SMAs of the same type, but having different properties, e.g. in terms of size, shape, etc. In this way, it is possible to tailor the SMAs of the same type to behave differently, e.g. by providing a high force-low speed SMA in combination with a low force-high speed SMA together.

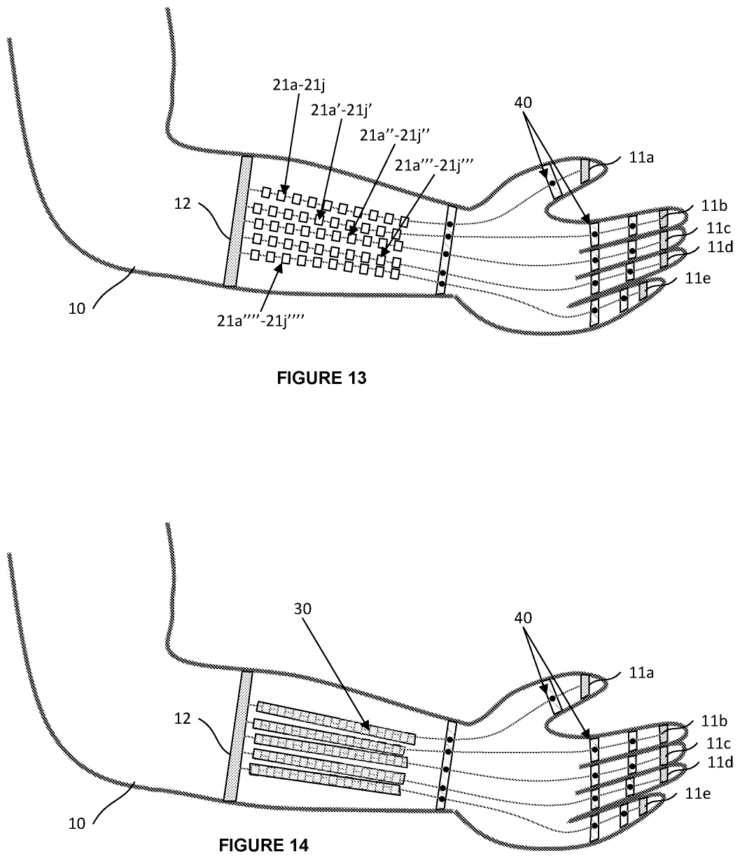

FIG. 13 illustrates an example where the garment 10 comprises a lower arm sleeve with a glove. A number of groups of in-sequence SMAs 21a-21j to 21a″″-21j′″ are connected between a respective first anchor zone 11a-11e arranged at a fingertip end of the glove and the second anchor zone 12 arranged around the lower arm part of the garment 10. At least part of the linkages of each group comprises a wire, e.g. a force translation wire, which is able to be slidably guided via one or more guide zones 20. Each guide zone 40 may comprise one or more guide members, e.g. channel, loops, or hoops, (shown as black dots in FIGS. 13 to 16) through which the force translation wire slidably extends. The guide zone(s) may be configured to prevent any undesired splay by guiding the wires along predefined directions, which in turn triggers, facilitates or resists the movement of the muscles of the person or animal wearing the garment assembly along said predefined directions. An end of the force translation wire may be attached to the respective first anchor zone 11a-11e, wherein the other end is attached to one the SMAs of the respective group.

In a preferred example, the smart actuator is a photo-responsive actuator. The photo-responsive actuator may be a photoactive polymer monomer, such as 2,4-dihydroxy-4-nitroazobenzene, or 2,4-dihydroxy-4-azo-(4-nitroazobenzeno) benzene. The present inventors have through various experiments realised that these photoactive polymer monomers provide for a suitable specific strength sufficient for scaling to a macroscale actuation, allowing for fast response rates, and/or enabling for any electronics to be safely isolated from the subject wearing the garment or garment assembly as they are activated by light.

Alternatively, or additionally, the photo responsive SMA material may also be selected from a photo-responsive or photo-active acrylate, such as an azobenzene monomer acrylate. For example, a 7-((4-((2-cyano-4-nitrophenyl)diazenyl)phenyl)(ethyl)amino)heptyl acrylate may be used.

Alternatively, or additionally, the photo responsive SMA material may also be selected from a photo responsive or photo active stilbene monomer, such as a di-stilbene monomer. For example, a 4,4′-((Propane-2,2-diylbis(4,1-phenylene)bis(Oxy)bis(4,1-phenylene))bis(ethylene-2,1-diyl))dianiline may be used.

For the preferred example, wherein the smart material actuators are of a photo-responsive type, the activation unit comprises a light source for transmitting light to activate each SMA.

In some examples, the activation unit comprises a number of light sources provided in a configuration, e.g. an array corresponding to that of the groups of SMAs. For example, the activation unit 40 of FIG. 14 comprise a number of LED strips, conforming with the shape of each group of SMAs of FIG. 13. In FIG. 13, the garment assembly comprises five LED strips. However, it should be appreciated that any number is possible. In some examples, the number of LED strips is less than the number of SMA groups, whereby each LED strip may be used to activate more than one SMA group.

The activation unit 30 may further comprise a light guide (as indicated by the arrows extending from the activation unit 30 in FIGS. 1 to 5) arranged to receive light from the said light source and direct said received light towards one or more SMAs.

Using light guides which are inherently flexible, it is possible to arrange the respective light guide at a predetermined distance, and at a predetermined orientation in relation to one or more SMAs when in the idle state.

In some examples, the light guides are physically detached from the respective group of in sequence SMAs.

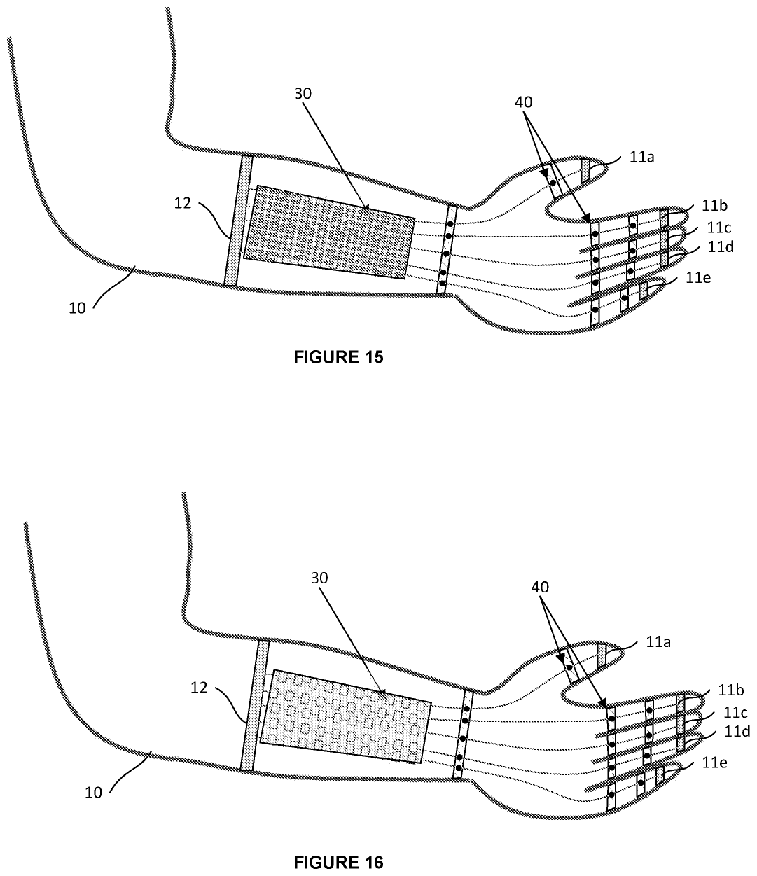

The activation unit 40 may comprise a Light Emitting Diode (LED) panel, such as a flexible LED panel, as shown with reference to FIG. 15.

In some configurations the LED panel or flexible LED panel may comprise at least one LED per SMA, wherein the one or more LEDs of the LED panel has a spatial configuration corresponding to that of the associated SMAs. However, in alternative examples the spatial configuration of the LEDs of the LED panel may differ from that of the SMAs. Hence, a one-to-one spatial configuration match between each SMA and a respective LED of the LED panel may not be required. Instead, one or more LEDs of the LED panel roughly covering any associated SMA may be activated for activation of the respective SMA, or vice versa.

In some configurations, the LED panel or flexible LED panel may comprise less LEDs than there are SMAs, whereby more than one SMA may be activated by a single LED.

Alternatively, the activation unit may comprise one or more LEDs attached to a flexible printed circuit board (PCB), that is arranged in close proximity to the associated photo responsive SMAS.

The one or more LEDs or the LED panel may be attached to the garment at the locations of associated SMAs. Alternatively, or additionally the SMAs could be activated remotely, such as via laser light.

In applications where the at least one SMA is a photo-responsive actuator, for activation, the activation unit is arranged to transmit a non-mechanical stimulus comprising light to the SMA. Here, the activated state of the SMA is triggered by receiving the light from the activation unit. The deactivated state of the SMA refers to a state where no light is transmitted to the SMA. Hence, to trigger the deactivated state, the activation unit is controlled to stop transmitting light.

In general, a photo-responsive material, such as one of those disclosed herein, will be inclined to return towards its original idle state, upon deactivation. If the SMA mechanically expands upon activation, its associated activated length will be increased (provided there is no external load preventing it from expanding or an external load working against the expansion) as compared to its length in the deactivated state. Upon deactivation from the activated state, the length of the SMA will decrease (provided there is no external load preventing it from contracting or an external load acting against the contraction) and return to its length in the deactivated state.

As a result of the mechanical contraction and/or mechanical expansion a force is created between the anchor zones. During mechanical expansion the associated force created will act to push the anchor zones further apart. During mechanical contraction the associated force created will act to pull the anchor zones towards each other. In other words, upon activation of the SMA(s), the created force will act on the respective anchor zones to which the SMA(s) are attached.

Factors or parameters affecting whether a photo responsive SMA acts to mechanically contract or mechanically expand upon activation (i.e. when receiving light) may include at least one the following:
the type of the SMA material and/or material composition thereof,
the wavelength(s) transmitted and received by the SMA,
the light intensity, and
the light polarisation, including associated polarisation angles.

In some examples, the LEDs of the LED panel may be arranged to transmit blue, ultraviolet and/or green light or any wavelength or combination of wavelengths ranging between 100 nm to 1000 nm. In some examples, the blue, ultraviolet and/or green LEDs are alternating over the LED panel.

As an example, for at least some of the photo-responsive actuators identified above, some wavelengths are particularly effective at causing chiral change, i.e. compound shape change.

Depending on the type of photo-responsive actuator selected shorter wavelengths such as ultraviolet (UV) wavelengths (<400 nm) or blue wavelengths in the end of the visual spectrum may cause a mechanical contraction. Longer wavelengths, such as green wavelengths (˜550 nm) may cause a mechanical expansion of the photo-responsive actuator. Wavelengths in between the UV and green, such as a range of blue wavelengths (400-550 nm) may cause mechanical contraction or expansion depending on the selected type of SMA. Emitting light in these wavelengths may allow for fine tuning the mechanical contraction and/or expansion in terms of percentage contraction. The selection of wavelengths also affects the response rate as some types of photo responsive SMAs are more responsive to particular wavelengths. Further, some wavelengths may be advantageous for causing a long-term shape change of the associated SMA while other wavelengths allow for only a short-term shape change of the associated SMA after the activation unit emitting electromagnetic radiation using said wavelengths has been deactivated.

In an example, as illustrated with reference to FIG. 16, the activation unit 30 is a luminescent film or panel.

In some examples, the light source may be an external light source being remotely arranged. For example, the light source may be a laser.

Alternatively, an electroluminescent paint forming part of the activator unit may be used to form a thin film light source, wherein light is emitted when the paint is electrically stimulated by electrodes.

Other examples of thin light sources may be one or more light guide(s) coupled with fibre optics that is then coupled to one or more lasers or LEDs. Alternatively, or additionally, the thin film may comprise quantum dots embedded therein. The quantum dots may be electrically powered, e.g. using the controller of the activation unit, to produce light which in turn is used to activate the SMAs. Alternatively, or additionally, bioluminescence where light is emitted by living organisms or chemiluminescence where light is produced as a result of chemical change/reaction could also be used as a light source.

In alternative examples, the SMA may also be selected from the group consisting of Dielectric or electro restrictive elastomer actuators (DEA), Conductive polymer actuators (CP), Electroactive polymer actuators (EAP), Magneto strictive actuators (MA), shape memory polymers (SMP), such as light activated SMPs (LASMP).

In some examples, the activation unit 30 may be releasably or fixedly attached to the garment. Arranging the activation unit 30 in a fixed relation to the respective SMAs may be advantageous for some types of SMAs, e.g. photo responsive SMAs, where the overall efficiency is improved when light is directed towards each SMA from at a certain angle and intensity.

For example, the activator unit may be provided in a location that minimizes bulk and disruption to movement, e.g. attached onto a belt of the subject wearing the garment or garment assembly, or on the arm of the subject wearing the garment or garment assembly.

In some examples, the activation unit 30 is arranged distinct from the garment. For example, the activation unit may be arranged to be secured to a body part of the subject wearing the garment or garment assembly.

In some examples, activation of the SMAs may also allow for force feedback to the person wearing the garment or garment assembly. For example, in some applications the garment may be used as a virtual reality (VR) garment. Upon detection of a virtual object collision, the activation unit controlling the activation of the associated SMAs may be controlled (e.g. using a controller) to maintain a constant longitudinal distance even if the person wearing the garment or garment assembly actively attempts to move the limb onto which the garment is secured. Using sensor data from a sensor, as those described above, e.g. accelerometers, the limb movement may be detected and accessed by the controller. Upon accessing the sensor data, the controller may be configured to activate the SMAs so that the longitudinal distance of the force translation mechanism is maintained substantially constant. This will produce a force, working against the force of the person, that is experienced by the person as force feedback. For example, assuming the garment is a VR glove, and the person wearing a garment holds a virtual apple in his/her hand. Upon touching the apple, the controller would detect an object collision. The associated SMAs are activated, providing force feedback that makes it feel like the person is actually holding an apple. In the event the person would try to close his/her hand, the controller based on sensor input information would prevent such movement by adapting the control signal to the activation unit, maintaining the associated longitudinal distance of the force translation mechanism constant or substantially constant, whereby the person would experience a higher force, just like in real life.

As an alternative of controlling the SMAs to maintaining a constant longitudinal distance, the longitudinal distance may be controlled based on controlling the SMAs based on actions or events occurring, or objects encountered in the virtual world, such in a computer game. As an example, still assuming the garment assembly has a portion, such as a glove portion as depicted in FIGS. 1 to 5, and 8 to 16, and turning back to the apple example above, imagine the apple exploding after being held in the character's hand, upon the explosion the longitudinal distance may be altered (e.g. increased) for the user to get a sense of the explosion. Alternatively, imagining an example where the apple while gripped decays or shrinks in the character's hand, this could lead to an opposite change in the longitudinal distance, providing the person wearing the garment assembly with a sense that the apple is getting smaller or less dense. Hence, in some examples the SMAs may be controlled by events or actions in the virtual world, such as in a computer game, to provide the user with force feedback to make the virtual experience even more immersive.

The electrical components of the garment assembly, e.g. the controller and/or the activation unit may be powered by a power or battery pack optionally worn by the subject wearing the garment or garment assembly. In some examples, the power pack is configured to be attached to the garment. The power pack may comprise one or more batteries, e.g. rechargeable batteries. Any conventional battery could be used, e.g. lithium-ion batteries, etc.

In some examples, the garment assembly may comprise at least one solar panel operatively coupled to the power or battery pack or directly to the controller and/or activation unit.

Alternatively, and or additionally, the electrical circuitry (including the controller and activation unit, etc) of the garment assembly may be powered by a non-portable energy source, such as the power grid. In some examples, the garment assembly comprises a power cable for plugging into the power grid, such as via a regular wall socket or the like.

In some examples, the garment assembly further comprises a Human Machine Interface (HMI) connected to the controller that allows the user or subject wearing the garment or garment assembly to interface with the controller. The HMI may comprise a power ON/OFF function. Alternatively, or additionally the HMI interface may comprise a wireless communications interface, e.g. Bluetooth, WIFI or NFC allowing the user or clinician to control the operation of the controller. For example, a clinician may adjust parameters such as range of motion and resistance to using smartphone app or PC operatively coupled to the wireless communications interface.

The HMI may comprise physical buttons, or touch buttons to control various operation modes or functionalities of the controller. The HMI could further comprise a display unit such as a touch screen allowing for displaying of information related to power state, battery life, or optionally biometric data (e.g. heartrate, step count) collected from various biometric sensors. LED indicators may also be integrated into the HMI to power state (ON/OFF) etc.

The activation sequence may be programmed to follow certain exercise movements for rehabilitation purposes. Alternatively, the activation sequence may be programmed by a_clinician or patient to fine-tune biomechanical parameters to tailor to each patient need for active mobility purposes. The activation sequence may also be programmed to assist the subject's own movements to assist in lifting exercises etc. For example, the controller may be programmed to allow for adjustment of the amount of assistance required, according to a scale such as between 0% and 100%, such as via a smartphone application connected to the wireless communications interface.

While the examples above are described with reference to a garment assembly suitable for facilitating or resisting movement of a subject, i.e. a human, wearing the garment assembly, it should be appreciated that the garment assembly alternatively could be adapted and designed for facilitating or resisting movement of any animal (such as for veterinary or rehab purposes), wherein the associated garment is tailored to said animal. In other words, the configuration of the SMAs, anchor zones, and associated force translation mechanism of the garment assembly disclosed herein may act as an artificial muscle triggering, facilitating, or resisting muscle movement of the subject wearing the garment assembly.

Further, the garment assembly may also be used for facilitating or resisting movement in an inanimate object or being, such as robot or toy, when the garment assembly is worn by the inanimate object or being. In this way, the configuration of the SMAs, anchor zones, and associated force translation mechanism of the garment assembly disclosed herein may act as an artificial muscle triggering, facilitating, or resisting optional actuator movement of the inanimate object wearing the garment assembly.

The garment assembly may be designed (in terms and size and shape) to be worn by any animate or inanimate being for facilitating or resisting movement.

In some examples, the respective anchor zones, SMAs, and associated force translation mechanism form an actuator assembly. Hence, rather than the garment comprising the respective anchor zones, SMAs, and associated force translation mechanism, the actuator assembly may be provided as a separate assembly, distinct from the garment. The actuator assembly may be arranged to be attached to garment to form the garment assembly. The actuator assembly may be attached to the garment at the location of or via the respective anchor zones. The actuator assembly may further comprise the activation unit.

### CLAUSES

Various examples of the present disclosure will now be explained with reference to the following clauses.
Clause 1. A garment assembly for facilitating or resisting movement of a subject, such as a person, animal, or inanimate object, wearing the garment assembly, comprising
   a garment having a first anchor zone, a second anchor zone, each anchor zone acting to secure the garment to a respective body part of the subject, in use,
   at least two first smart material actuators (SMAs) connected in sequence between the first anchor zone and second anchor zone, and forming a first group of SMAS,
   a force translation mechanism connecting the first anchor zone to the second anchor zone and comprising the first group of SMAs,
   wherein each SMA is arranged to operate in an idle state, and an activated state triggered by a non-mechanical stimulus that causes the associated SMA to mechanically contract, and
   an activation unit arranged to transmit said non-mechanical stimulus to each SMA individually in response to a defined activation sequence.
Clause 2. A garment assembly for facilitating or resisting movement of a subject, such as a person, animal, or inanimate object, wearing the garment assembly, comprising
   a garment having a first anchor zone, a second anchor zone, each anchor zone acting to secure the garment to a respective body part of the subject, in use,
   at least two first smart material actuators (SMAs) connected in sequence between the first anchor zone and second anchor zone, and forming a first group of SMAs,
   a force translation mechanism connecting the first anchor zone to the second anchor zone and comprising the first group of SMAS,
   wherein each SMA is arranged to operate in an idle state, and an activated state triggered by a non-mechanical stimulus that causes the associated SMA to mechanically expand, and
   an activation unit arranged to transmit said non-mechanical stimulus to each SMA individually in response to a defined activation sequence.
Clause 3. The garment assembly according to clause 1 or 2, wherein the defined activation sequence is associated with a desired movement and/or orientation of a body part of the subject wearing the garment.
Clause 4. The garment assembly according to any one of clauses 1 to 3, further comprising a fabric, to which the first anchor zone and the second anchor zone is fixedly attached.
Clause 5. The garment assembly according to clause 4, wherein each SMA is fixedly or releasably attached to the fabric.
Clause 6. The garment assembly according to any one of the preceding clauses, wherein the force translation mechanism comprises one or more linkages, wherein each linkage comprises at least one of a fibre, yarn, cable, wire, film, or strip.
Clause 7. The garment assembly according to clause 6, wherein the one or more linkages has a material being at least to an extent partly rigid, non-flexible, or inelastic.
Clause 8. The garment assembly according to any one of the preceding clauses, wherein the force translation mechanism comprises a first end anchored to the first anchor zone or second anchor zone and a second end attached to the at least one of the in-sequence SMAs.
Clause 9. The garment assembly according to any one of clauses 6 to 8, wherein the force translation mechanism comprises at least one of:
   a first linkage connected between and attached to the first anchor zone and an SMA closest in sequence to the first anchor zone,
   a second linkage connected between and attached to two closest neighbouring SMAs, and
   a third linkage connected between and attached to the second anchor zone and a SMA closest in sequence to the second anchor zone.
Clause 10. The garment assembly according to any one of the preceding clauses, wherein the at least two SMAs connected in sequence form part of a laterally detached or laterally attached SMA array comprising more than one group of SMAs.
Clause 11. The garment assembly according to any of the preceding clauses, wherein the first anchor zone and/or the second anchor zone comprises at least one second SMA, arranged to mechanically contract in its activated state to secure the garment to the respective body part of the subject or to provide force feedback.
Clause 12. The garment assembly according to any of the preceding clauses, wherein each SMA is selected from the group consisting of:
   Photo-responsive actuators;
   Dielectric or electro restrictive elastomer actuators (DEA);
   Conductive polymer actuators (CP);
   Electroactive polymer actuators (EAP);
   Magneto strictive actuators (MA); and
   Shape memory polymers (SMP).
Clause 13. The garment assembly according to clause 10 or any clause dependent thereon, wherein the SMA array is provided as a thin film array configured to conform around a curvature of the human body.
Clause 14. The garment assembly according to clause 13, wherein the thin film comprises a clear coating or plastic film applied over the array of SMAs, wherein the clear coating or plastic film is arranged to translate actuation forces through its structure.
Clause 15. The garment assembly according to any one of the preceding clauses, wherein the first group of SMAs are connected in sequence along a first direction between first anchor zone and the second anchor zone.
Clause 16. The garment assembly according to clause 15, wherein the first group of SMAs are formed in an array comprising at least one second group of in-sequence connected SMAs arranged in parallel to the first direction.
Clause 17. The garment assembly according to clause 15 or 16, further comprising at least two further smart material actuator(s) (SMAs) connected in sequence between the first anchor zone and second anchor zone, and forming a second group of SMAs, wherein the second group of SMAs connected in sequence along a second direction between first anchor zone and the second anchor zone, wherein the first direction is different from the second direction.
Clause 18. The garment assembly according to clause 17, wherein the second group of SMAs are formed in an array comprising at least one further second group of in-sequence connected SMAs arranged in parallel to the second direction.
Clause 19. The garment assembly according to clause 17 or 18, wherein the first group of SMAs are arranged in a first layer of the garment, and the second group of SMAs are arranged in a second layer of the garment, wherein the second layer at least partly overlaps the first layer.
Clause 20. The garment assembly according to any one of the preceding clauses, wherein the activation unit is releasably or fixedly attached to the garment.
Clause 21. The garment assembly according to any one of clauses 1 to 19, wherein the activation unit is arranged to be attached to a body part of the subject, wearing the garment, in use.
Clause 22. The garment assembly according to any one of the preceding clauses, wherein at least one of the SMAs is a photo-responsive actuator, and wherein the
   activation unit comprises a light source for transmitting light to activate at least part of the at least one SMA.
Clause 23. The garment assembly according to clause 22, wherein the activation unit further comprises a light guide arranged to receive light from the said light source and direct said received light towards said at least one associated SMA.
Clause 24. The garment assembly according to clause 22 or 23, wherein the activation unit comprises a Light Emitting Diode (LED) panel comprising at least one LED per SMA, wherein the one or more LEDs of the LED panel has a spatial configuration corresponding to that of the associated SMAs.
Clause 25. The garment assembly according to any one of clauses 22 to 24, wherein the activation unit comprises a flexible Light Emitting Diode (LED) panel comprising at least one LED per SMA, wherein the one or more LEDs of the flexible LED panel has a spatial configuration corresponding to that of the associated SMAs.
Clause 26. The garment assembly according to any one of clauses 22 to 24, wherein the activation unit comprises one or more LEDs attached to a flexible printed circuit board (PCB).
Clause 27. The garment assembly according to any one of clauses 22 to 24, wherein the activation unit comprises one or more LEDs attached to the garment for activating the associated SMAs.
Clause 28. A garment assembly for facilitating or resisting movement of a subject, such as a person, animal, or inanimate object, wearing the garment, comprising
   a garment having
      a first anchor zone, a second anchor zone, each anchor zone acting to secure the garment to a respective body part of the subject, in use,
      at least one first smart material actuator (SMAs) connected between the first anchor zone and second anchor zone, and forming a first group of SMAS,
      a force translation mechanism connecting the first anchor zone to the second anchor zone and comprising the first group of SMAS,
      wherein the first anchor zone and/or the second anchor zone comprises at least one second SMA,
      wherein each SMA is arranged to operate in an idle state, and an activated state triggered in response to a non-mechanical stimulus that causes the associated SMA to mechanically contract, wherein said at least second SMA is arranged to mechanically contract in its activated state to secure the garment to the respective body part of the subject or to provide force feedback, and
   an activation unit arranged to transmit said non-mechanical stimulus to each SMA individually in response to a defined activation sequence.
Clause 29. A garment assembly for facilitating or resisting movement of a subject, such as a person, animal, or inanimate object, wearing the garment, comprising
   a garment having
      a first anchor zone, a second anchor zone, each anchor zone acting to secure the garment to a respective body part of the subject, in use,
      at least one first smart material actuator (SMAs) connected between the first anchor zone and second anchor zone, and forming a first group of SMAs,
      a force translation mechanism connecting the first anchor zone to the second anchor zone and comprising the first group of SMAs,
      wherein the first anchor zone and/or the second anchor zone comprises at least one second SMA,
      wherein each SMA is arranged to operate in an idle state, and an activated state triggered in response to a non-mechanical stimulus that causes the associated SMA to mechanically expand, wherein said at least second SMA is arranged to mechanically expand in its activated state to secure the garment to the respective body part of the subject or to provide force feedback, and
   an activation unit arranged to transmit said non-mechanical stimulus to each SMA individually in response to a defined activation sequence.
Clause 30. The garment assembly according to clause 28 or 29, wherein the first group of SMAs comprises at least two first smart material actuator(s) (SMAs) connected in sequence between the first anchor zone and second anchor zone.
Clause 31. The garment assembly according to any one of clauses 28 to 30, wherein the defined activation sequence is associated with a desired movement and/or orientation of a body part of the subject wearing the garment.
Clause 32. The garment assembly according to any one of clauses 28 to 31, further comprising a fabric, to which the first anchor zone and the second anchor zone is fixedly attached.
Clause 33. The garment assembly according to clause 32, wherein each SMA is fixedly or releasably attached to the fabric.
Clause 34. The garment assembly according to any one of clauses 28 to 33, wherein the force translation mechanism comprises one or more linkages, wherein each linkage comprises at least one of a fibre, yarn, cable, wire, film, or strip.
Clause 35. The garment assembly according to clause 34, wherein the one or more linkages has a material being at least to an extent partly rigid, non-flexible, or inelastic.
Clause 36. The garment assembly according to any one of clauses 28 to 35, wherein the force translation mechanism comprises an elastomeric material with a first end anchored onto the first anchor zone and a second end attached to the at least one of the in-sequence SMAs.
Clause 37. The garment assembly according to any one of clauses 28 to 36, wherein the force translation mechanism comprises at least:
   a first linkage connected between and attached to the first anchor zone and an SMA closest in sequence to the first anchor zone,
   a second linkage connected between and attached to two closest neighbouring SMAs, and
   a third linkage connected between and attached to the second anchor zone and a SMA closest in sequence to the second anchor zone.
Clause 38. The garment assembly according to any one of clauses 28 to 37, wherein the at least two SMAs connected in sequence form part of a laterally detached or laterally attached SMA array comprising more than one group of SMAs.
Clause 39. The garment assembly according to any of the clauses 28 to 38, wherein each SMA is selected from the group consisting of:
   Photo-responsive actuators;
   Dielectric or electro restrictive elastomer actuators (DEA);
   Conductive polymer actuators (CP);
   Electroactive polymer actuators (EAP);
   Magneto strictive actuators (MA); and
   Shape memory polymers (SMP).
Clause 40. The garment assembly according to clause 38 or any clause dependent thereon, wherein the SMA array is provided as a thin film array configured to conform around a curvature of the human body.
Clause 41. The garment assembly according to clause 40, wherein the thin film comprises a clear coating or plastic film applied over the array of SMAs, wherein the clear coating or plastic film is arranged to translate actuation forces through its structure.
Clause 42. The garment assembly according to any one of clauses 28 to 42, wherein the first group of SMAs are connected in sequence along a first direction between first anchor zone and the second anchor zone.
Clause 43. The garment assembly according to clause 42, wherein the first group of SMAs are formed in an array comprising at least one further first group of in-sequence connected SMAs arranged in parallel to the first direction.
Clause 44. The garment assembly according to clause 42 or 43, further comprising at least two further smart material actuator(s) (SMAs) connected in sequence between the first anchor zone and second anchor zone, and forming a second group of SMAs, wherein the second group of SMAs connected in sequence along a second direction between first anchor zone and the second anchor zone, wherein the first direction is different from the second direction.
Clause 45. The garment assembly according to clause 44, wherein the second group of SMAs are formed in an array comprising at least one further second group of in-sequence connected SMAs arranged in parallel to the second direction.
Clause 46. The garment assembly according to clause 44 or 45, wherein the first group of SMAs are arranged in a first layer of the garment, and the second group of SMAs are arranged in a second layer of the garment, wherein the second layer at least partly overlaps the first layer.
Clause 47. The garment assembly according to any one of clauses 28 to 46, wherein the activation unit is releasably or fixedly attached to the garment.
Clause 48. The garment assembly according to any one of clauses 28 to 46, wherein the activation unit is arranged to a body part comprising the garment.
Clause 49. The garment assembly according to any one of clauses 28 to 48, wherein at least one of the SMAs is a photo-responsive actuator, and wherein the
   activation unit comprises a light source for transmitting light to activate at least part of the at least one SMA.
Clause 50. The garment assembly according to clause 49, wherein the activation unit further comprises a light guide arranged to receive light from the said light source and direct said received light towards said at least one associated SMA.
Clause 51. The garment assembly according to clause 49 or 50, wherein the activation unit comprises a Light Emitting Diode (LED) panel comprising at least one LED per SMA, wherein the one or more LEDs of the LED panel has a spatial configuration corresponding to that of the associated SMAs.
Clause 52. The garment assembly according to any one of clauses 49 to 51, wherein the activation unit comprises a flexible Light Emitting Diode (LED) panel comprising at least one LED per SMA, wherein the one or more LEDs of the flexible LED panel has a spatial configuration corresponding to that of the associated SMAs.
Clause 53. The garment assembly according to any one of clauses 49 to 51, wherein the activation unit comprises one or more LEDs attached to a flexible printed circuit board (PCB).
Clause 54. The garment assembly according to any one of clauses 49 to 51, wherein the activation unit comprises one or more LEDs attached to the garment for activating the associated SMAs.
Clause 55. A garment assembly for facilitating or resisting movement of a subject, such as a person, animal, or inanimate object, wearing the garment, comprising
   a garment having
      a first anchor zone, a second anchor zone, each anchor zone acting to secure the garment to a respective body part of the subject, in use,
      at least one first smart material actuator(s) (SMAs) arranged between the first anchor zone and second anchor zone, and oriented along a first direction of the garment, and forming a first group of SMAs,
      at least one second smart material actuator(s) (SMAs) arranged between the first anchor zone and second anchor zone, and oriented along a second direction of the garment, and forming a second group of SMAs, wherein the first direction is different from the second direction,
      a force translation mechanism connecting the first anchor zone to the second anchor zone and comprising the first group of SMAs, and second group of SMAs,
      wherein each SMA is arranged to operate in an idle state, and an activated state triggered in response to a non-mechanical stimulus that causes the associated SMA to mechanically contract, and
   an activation unit arranged to transmit said non-mechanical stimulus to each SMA individually in response to a defined activation sequence.
Clause 56. A garment assembly for facilitating or resisting movement of a subject, such as a person, animal, or inanimate object, wearing the garment, comprising
   a garment having
      a first anchor zone, a second anchor zone, each anchor zone acting to secure the garment to a respective body part of the subject, in use,
      at least one first smart material actuator(s) (SMAs) arranged between the first anchor zone and second anchor zone, and oriented along a first direction of the garment, and forming a first group of SMAS,
      at least one second smart material actuator(s) (SMAs) arranged between the first anchor zone and second anchor zone, and oriented along a second direction of the garment, and forming a second group of SMAs, wherein the first direction is different from the second direction,
      a force translation mechanism connecting the first anchor zone to the second anchor zone and comprising the first group of SMAs, and second group of SMAS,
      wherein each SMA is arranged to operate in an idle state, and an activated state triggered in response to a non-mechanical stimulus that causes the associated SMA to mechanically expand, and
   an activation unit arranged to transmit said non-mechanical stimulus to each SMA individually in response to a defined activation sequence.
Clause 57. The garment assembly according to clause 55 or 56, wherein the first group of SMAs comprises at least two first smart material actuator(s) (SMAs) connected in sequence between the first anchor zone and second anchor zone.
Clause 58. The garment assembly according to any one of clauses 55 to 57, wherein the second group of SMAs comprises at least two first smart material actuator(s) (SMAs) connected in sequence between the first anchor zone and second anchor zone.
Clause 59. The garment assembly according to any one of clauses 55 to 58, wherein the defined activation sequence is associated with a desired movement and/or orientation of a body part of the subject wearing the garment.
Clause 60. The garment assembly according to clause any one of clauses 55 to 59,further comprising a fabric, to which the first anchor zone and the second anchor zone is fixedly attached.
Clause 61. The garment assembly according to clause 60, wherein each SMA is fixedly or releasably attached to the fabric.
Clause 62. The garment assembly according to any one of clauses 55 to 61, wherein the force translation mechanism comprises one or more linkages, wherein each linkage comprises at least one of a fibre, yarn, cable, wire, film, or strip.
Clause 63. The garment assembly according to clause 62, wherein the one or more linkages has a material being at least to an extent partly rigid, non-flexible, or inelastic.
Clause 64. The garment assembly according to any one of clauses 55 to 63, wherein the force translation mechanism comprises an elastomeric material with a first end anchored onto the first anchor zone and a second end attached to the at least one of the in-sequence SMAs.
Clause 65. The garment assembly according to any one of clauses 62 to 64, wherein the force translation mechanism comprises at least:
   a first linkage connected between and attached to the first anchor zone and an SMA closest in sequence to the first anchor zone,
   a second linkage connected between and attached to two closest neighbouring SMAs, and
   a third linkage connected between and attached to the second anchor zone and a SMA closest in sequence to the second anchor zone.
Clause 66. The garment assembly according to clause 57 or 58, or any clause dependent thereon, wherein the at least two SMAs connected in sequence form part of a laterally detached or laterally attached SMA array comprising more than one group of SMAs.
Clause 67. The garment assembly according to any of clauses 55 to 66, wherein the first anchor zone and/or the second anchor zone comprises at least one second SMA, arranged to mechanically contract in its activated state to secure the garment to the respective body part of the subject or to provide force feedback.
Clause 68. The garment assembly according to any of clauses 55 to 67, wherein each SMA is selected from the group consisting of:
   Photo-responsive actuators;
   Dielectric or electro restrictive elastomer actuators (DEA);
   Conductive polymer actuators (CP);
   Electroactive polymer actuators (EAP);
   Magneto strictive actuators (MA); and
   Shape memory polymers (SMP).
Clause 69. The garment assembly according to clause 66 or any clause dependent thereon, wherein the SMA array is provided as a thin film array configured to conform around a curvature of the human body.
Clause 70. The garment assembly according to clause 69, wherein the thin film comprises a clear coating or plastic film applied over the array of SMAs, wherein the clear coating or plastic film is arranged to translate actuation forces through its structure.
Clause 71. The garment assembly according to clause 55 or 56, or any clause dependent thereon, wherein the first group of SMAs are formed in an array comprising at least one further first group of in-sequence connected SMAs arranged in parallel to the first direction.
Clause 72. The garment assembly according to clause 55 or 56, or any clause dependent thereon, wherein the second group of SMAs are formed in an array comprising at least one further second group of in-sequence connected SMAs arranged in parallel to the second direction.
Clause 73. The garment assembly according to any one of clauses 55 to 72, wherein the first group of SMAs are arranged in a first layer of the garment, and the second group of SMAs are arranged in a second layer of the garment, wherein the second layer at least partly overlaps the first layer.
Clause 74. The garment assembly according to any one of clauses 55 to 73, wherein the activation unit is releasably or fixedly attached to the garment.
Clause 75. The garment assembly according to any one of clauses 55 to 73, wherein the activation unit is arranged to a body part comprising the garment.
Clause 76. The garment assembly according to any one of clauses 55 to 75, wherein at least one of the SMAs is a photo-responsive actuator, and wherein the
   activation unit comprises a light source for transmitting light to activate at least part of the at least one SMA.
Clause 77. The garment assembly according to clause 76, wherein the activation unit further comprises a light guide arranged to receive light from the said light source and direct said received light towards said at least one associated SMA.
Clause 78. The garment assembly according to clause 76 or 77, wherein the activation unit comprises a Light Emitting Diode (LED) panel comprising at least one LED per SMA, wherein the one or more LEDs of the LED panel has a spatial configuration corresponding to that of the associated SMAs.
Clause 79. The garment assembly according to any one of clauses 76 to 78, wherein the activation unit comprises a flexible Light Emitting Diode (LED) panel comprising at least one LED per SMA, wherein the one or more LEDs of the flexible LED panel has a spatial configuration corresponding to that of the associated SMAs.
Clause 80. The garment assembly according to any one of clauses 76 to 78, wherein the activation unit comprises one or more LEDs attached to a flexible printed circuit board (PCB).
Clause 81. The garment assembly according to any one of clauses 76 to 78, wherein the activation unit comprises one or more LEDs attached to the garment for activating the associated SMAs.
Clause 82. A garment assembly for facilitating or resisting movement of a subject, such as a person, animal, or inanimate object, wearing the garment assembly, comprising
   a garment having
      a first anchor zone, a second anchor zone, each anchor zone acting to secure the garment to a respective body part of the subject, in use,
      at least one smart material actuators (SMAs) connected between the first anchor zone and second anchor zone, and forming a first group of SMAs,
      a force translation mechanism connecting the first anchor zone to the second anchor zone and comprising the first group of SMAs,
      wherein each SMA is arranged to operate in an idle or deactivated state, and an activated state triggered by a non-mechanical stimulus that causes a geometrical change in the associated SMA, and
   an activation unit arranged to transmit said non-mechanical stimulus to each SMA individually in response to a defined activation sequence.
Clause 83. A garment assembly for facilitating or resisting movement of a subject, such as a person, animal, or inanimate object, wearing the garment assembly, comprising
   a garment having
      a first anchor zone, a second anchor zone, each anchor zone acting to secure the garment to a respective body part of the subject, in use,
      at least one smart material actuators (SMAs) connected between the first anchor zone and second anchor zone, and forming a first group of SMAS,
      a force translation mechanism connecting the first anchor zone to the second anchor zone and comprising the first group of SMAS,
      wherein each SMA is arranged to operate in an idle or deactivated state, and an activated state triggered by a non-mechanical stimulus that causes the respective activated SMA to create a force acting on the first anchor zone and second anchor zone, and
   an activation unit arranged to transmit said non-mechanical stimulus to each SMA individually in response to a defined activation sequence.
Clause 84. A garment assembly for facilitating or resisting movement of a subject, such as a person, animal, or inanimate object, wearing the garment assembly, comprising
   a garment, and
   an actuator assembly comprising
      a first anchor zone, a second anchor zone, each anchor zone acting to secure the garment to a respective body part of the subject, in use,
      at least one smart material actuators (SMAs) connected between the first anchor zone and second anchor zone, and forming a first group of SMAs,
      a force translation mechanism connecting the first anchor zone to the second anchor zone and comprising the first group of SMAS,
      wherein each SMA is arranged to operate in an idle or deactivated state, and an activated state triggered by a non-mechanical stimulus that causes a geometrical change in the associated SMA, and
   an activation unit arranged to transmit said non-mechanical stimulus to each SMA individually in response to a defined activation sequence.
Clause 85. The garment assembly according to clause 8284, wherein the first group of SMAs comprises at least two first smart material actuator(s) (SMAs) connected in sequence between the first anchor zone and second anchor zone.
Clause 86. The garment assembly according to any one of clauses 82 to 85, wherein the second group of SMAs comprises at least two first smart material actuator(s) (SMAs) connected in sequence between the first anchor zone and second anchor zone.
Clause 87. The garment assembly according to any one of clauses 82 to 86, wherein the defined activation sequence is associated with a desired movement and/or orientation of a body part of the subject wearing the garment.
Clause 88. The garment assembly according to clause any one of clauses 82 to 87, further comprising a fabric, to which the first anchor zone and the second anchor zone is fixedly attached.
Clause 89. The garment assembly according to clause 88, wherein each SMA is fixedly or releasably attached to the fabric.
Clause 90. The garment assembly according to any one of clauses 82 to 89, wherein the force translation mechanism comprises one or more linkages, wherein each linkage comprises at least one of a fibre, yarn, cable, wire, film, or strip.
Clause 91. The garment assembly according to clause 90, wherein the one or more linkages has a material being at least to an extent partly rigid, non-flexible, or inelastic.
Clause 92. The garment assembly according to any one of clauses 82 to 91, wherein the force translation mechanism comprises an elastomeric material with a first end anchored onto the first anchor zone and a second end attached to the at least one of the in-sequence SMAs.
Clause 93. The garment assembly according to any one of clauses 90 to 92, wherein the force translation mechanism comprises at least:
   a first linkage connected between and attached to the first anchor zone and an SMA closest in sequence to the first anchor zone,
   a second linkage connected between and attached to two closest neighbouring SMAs, and
   a third linkage connected between and attached to the second anchor zone and a SMA closest in sequence to the second anchor zone.
Clause 94. The garment assembly according to clause 85 or 86, or any clause dependent thereon, wherein the at least two SMAs connected in sequence form part of a laterally detached or laterally attached SMA array comprising more than one group of SMAs.
Clause 95. The garment assembly according to any of clauses 82 to 94, wherein the first anchor zone and/or the second anchor zone comprises at least one second SMA, arranged to mechanically contract in its activated state to secure the garment to the respective body part of the subject or to provide force feedback.
Clause 96. The garment assembly according to any of clauses 82 to 95, wherein each SMA is selected from the group consisting of:
   Photo-responsive actuators;
   Dielectric or electro restrictive elastomer actuators (DEA);
   Conductive polymer actuators (CP);
   Electroactive polymer actuators (EAP);
   Magneto strictive actuators (MA); and
   Shape memory polymers (SMP).
Clause 97. The garment assembly according to clause 94 or any clause dependent thereon, wherein the SMA array is provided as a thin film array configured to conform around a curvature of the human body.
Clause 98. The garment assembly according to clause 97, wherein the thin film comprises a clear coating or plastic film applied over the array of SMAs, wherein the clear coating or plastic film is arranged to translate actuation forces through its structure.
Clause 99. The garment assembly according to any one of clauses 82 to 84, or any clause dependent thereon, wherein the first group of SMAs are formed in an array comprising at least one further first group of in-sequence connected SMAs arranged in parallel to the first direction.
Clause 100. The garment assembly according to any one of clauses 82 to 84, or any clause dependent thereon, wherein the second group of SMAs are formed in an array comprising at least one further second group of in-sequence connected SMAs arranged in parallel to the second direction.
Clause 101. The garment assembly according to any one of clauses 82 to 100, wherein the first group of SMAs are arranged in a first layer of the garment, and the second group of SMAs are arranged in a second layer of the garment, wherein the second layer at least partly overlaps the first layer.
Clause 102. The garment assembly according to any one of clauses 82 to 101, wherein the activation unit is releasably or fixedly attached to the garment.
Clause 103. The garment assembly according to any one of clauses 82 to 101, wherein the activation unit is arranged to a body part comprising the garment.
Clause 104. The garment assembly according to any one of clauses 82 to 103, wherein at least one of the SMAs is a photo-responsive actuator, and wherein the activation unit comprises a light source for transmitting light to activate at least part of the at least one SMA.
Clause 105. The garment assembly according to clause 104, wherein the activation unit further comprises a light guide arranged to receive light from the said light source and direct said received light towards said at least one associated SMA.
Clause 106. The garment assembly according to clause 104 or 105, wherein the activation unit comprises a Light Emitting Diode (LED) panel comprising at least one LED per SMA, wherein the one or more LEDs of the LED panel has a spatial configuration corresponding to that of the associated SMAs.
Clause 107. The garment assembly according to any one of clauses 104 to 106, wherein the activation unit comprises a flexible Light Emitting Diode (LED) panel comprising at least one LED per SMA, wherein the one or more LEDs of the flexible LED panel has a spatial configuration corresponding to that of the associated SMAs.
Clause 108. The garment assembly according to any one of clauses 105 to 106, wherein the activation unit comprises one or more LEDs attached to a flexible printed circuit board (PCB).
Clause 109. The garment assembly according to any one of clauses 105 to 106, wherein the activation unit comprises one or more LEDs attached to the garment for activating the associated SMAs.

1. A garment assembly for facilitating or resisting movement of a subject, wearing the garment assembly, comprising:
a garment,
a first anchor zone, a second anchor zone, each anchor zone acting to secure the garment to a respective body part of the subject, in use,
at least one smart material actuator (SMAs) connected between the first anchor zone and second anchor zone, and forming a first group of SMAs,
a force translation mechanism connecting the first anchor zone to the second anchor zone and comprising the first group of SMAs,
wherein each SMA is arranged to operate in an idle or deactivated state, and an activated state triggered by a non-mechanical stimulus that causes at least one of:
a) a physical material property change; and
b) a geometrical change in the associated SMA, and
an activation unit arranged to transmit said non-mechanical stimulus to each SMA in response to a defined activation sequence,
wherein at least one of the SMAs is a photo-responsive actuator, and
wherein the activation unit comprises a light source for transmitting light to activate at least part of the at least one SMA.

2-3. (canceled)

4. The garment assembly according to claim 1, wherein the at least one SMA is configured to mechanically contract or expand subject to receiving the non-mechanical stimulus.

5. The garment assembly according to claim 1, comprising at least two first smart material actuators (SMAs) connected in sequence between the first anchor zone and second anchor zone, and forming the first group of SMAs.

6. The garment assembly according to claim 1, wherein the activation unit is arranged to transmit said non-mechanical stimulus to each SMA individually.

7. The garment assembly according to claim 1, wherein the defined activation sequence is associated with a desired movement and/or orientation of a body part of the subject wearing the garment.

8-12. (canceled)

13. The garment assembly according to claim 1, wherein the force translation mechanism comprises at least one of:
a first linkage connected between and attached to the first anchor zone and an SMA closest in sequence to the first anchor zone,
a second linkage connected between and attached to two closest neighbouring SMAs, and
a third linkage connected between and attached to the second anchor zone and a SMA closest in sequence to the second anchor zone.

14. The garment assembly according to claim 1, wherein at least two SMAs are connected in sequence and form part of a laterally detached or laterally attached SMA array comprising more than one group of SMAs.

15. The garment assembly according to claim 1, wherein the first anchor zone and/or the second anchor zone comprises at least one second SMA arranged to mechanically contract in its activated state to secure the garment to the respective body part of the subject or to provide force feedback.

16-17. (canceled)

18. The garment assembly according to claim 14, wherein the SMA array is provided as a thin film array configured to conform around a curvature of the human body.

19. The garment assembly according to claim 14, wherein the SMA array comprises a thin film and wherein the thin film comprises a coating, a clear coating, or plastic film, wherein at least one of the coating, clear coating or plastic film is applied over the SMA array, wherein the thin film is arranged to translate actuation forces through its structure.

20. The garment assembly according to claim 1, wherein the first group of SMAs are either individual SMAs or SMAs connected in sequence, and wherein the first group of SMAs is along a first direction between first anchor zone and the second anchor zone and
wherein the first group of SMAs is formed in an array comprising at least one second group of individual or in-sequence connected SMAs, wherein the at least one second group of SMAs is arranged in parallel to the first direction.

21. (canceled)

22. The garment assembly according to claim 1, wherein the first group of SMAs are connected in sequence along a first direction between the first anchor zone and the second anchor zone, the garment assembly further comprising at least one second smart material actuator (SMAs) arranged between the first anchor zone and second anchor zone, and oriented along a second direction of the garment, and forming a second group of SMAs, wherein the first direction is different from the second direction.

23. (canceled)

24. The garment assembly according to claim 22, wherein the second group of SMAs are formed in an array comprising at least one further second group of SMAs arranged in parallel to the second direction.

25. The garment assembly according to claim 1, wherein the first group of SMAs are arranged in a first layer of the garment, and a second group of SMAs are arranged in a second layer of the garment, wherein the second layer at least partly overlaps the first layer, wherein the first group of SMAs are oriented along a first direction between the first anchor zone and the second anchor zone, and wherein the second group of SMAs comprises at least one second smart material actuator (SMA) arranged between the first anchor zone and second anchor zone, and oriented along a second direction of the garment, and forming a second group of SMAs, wherein the first direction is different from the second direction.

26. The garment assembly according to claim 1, wherein the activation unit is at least one of:
a) releasably or fixedly attached to the garment; and
b) arranged to be attached to a body part of the subject, wearing the garment, in use.

27-28. (canceled)

29. The garment assembly according to claim 1, wherein the activation unit further comprises a light guide arranged to receive light from the said light source and direct said received light towards said at least one associated SMA.

30. The garment assembly according to claim 1, wherein the activation unit comprises a Light Emitting Diode (LED) panel comprising at least one LED per SMA, wherein the one or more LEDs of the LED panel has a spatial configuration corresponding to that of the associated SMAs.

31. The garment assembly according to claim 1, wherein the activation unit comprises a flexible Light Emitting Diode (LED) panel comprising at least one LED per SMA, wherein the one or more LEDs of the flexible LED panel has a spatial configuration corresponding to that of the associated SMAs.

32. The garment assembly according to claim 1, wherein the activation unit comprises one or more LEDs attached to a flexible printed circuit board (PCB).

33. The garment assembly according to claim 1, wherein the activation unit comprises one or more LEDs attached to the garment for activating the associated SMAs.
